(19) United States
(12) Patent Application Publication
(10) Pub. No.: US 2026/0325064 A1
(43) Pub. Date: Oct. 22, 2026

(54) IMPLANTABLE MEDICAL DEVICE-BASED DETERMINATION OF SLEEP APNEA BURDEN

(71) Applicant: Medtronic, Inc., Minneapolis, MN (US)

(72) Inventors: Paul G. Krause, Mahtomedi, MN (US); Gautham Rajagopal, Minneapolis, MN (US); Leonardo Rapallini, Edina, MN (US); Yong K. Cho, Excelsior, MN (US); Anna J. Malin, Paradise Valley, AZ (US)

(21) Appl. No.: 19/640,999

(22) Filed: Apr. 7, 2026

Related U.S. Application Data
(60) Provisional application No. 63/790,315, filed on Apr. 17, 2025.

(51) Int. Cl. A61B 5/00; A61B 5/0245; A61B 5/29; A61B 5/346

(52) U.S. Cl. CPC A61B 5/4818; A61B 5/0245; A61B 5/29; A61B 5/346; A61B 5/4833; A61B 5/686; A61B 5/7264

(57) ABSTRACT

An example medical device includes sensing circuitry configured to sense a cardiac signal indicating activity of a heart of a patient; and processing circuitry configured to: detect one or more sleep apnea episodes of the patient occurring during a period of time based on the cardiac signal; compare an amount of detected sleep apnea episodes of the patient during the period of time to a sleep apnea severity threshold; and determine a classification of sleep apnea burden of the patient based on the comparison.

### RELATED APPLICATIONS

This application claims the benefit of U.S. Provisional Application Serial Number 63/790,315, filed Apr. 17, 2025, the entire contents of which are incorporated herein by reference.

### TECHNICAL FIELD

This disclosure relates to techniques for detecting patient sleep apnea episodes and monitoring patient health.

### BACKGROUND

When functioning properly, a heart maintains its own intrinsic rhythm and is capable of pumping adequate blood throughout a circulatory system. This intrinsic rhythm is a function of intrinsic signals generated by the sinoatrial (SA) node located in the upper right atrium. The SA node periodically depolarizes, which in turn causes the atrial heart tissue to depolarize so that right and left atria contract as the depolarization travels through the atrial heart tissue. The atrial depolarization signal is also received by the atrioventricular node, or AV node, which, in turn, triggers a subsequent ventricular depolarization signal that travels through and depolarizes the ventricular heart tissue causing the right and left ventricles to contract.

A condition known as sleep apnea can diminish cardiac output and pose various risks to patients, particularly those who are susceptible to heart failure. Sleep apnea is a sleep disorder that involves the temporary cessation of respiratory airflow during sleep. In various scenarios, sleep apnea may be characterized by one or both of pauses in breathing or periods of shallow breathing during sleep. Sleep apnea is generally considered a medical syndrome that occurs in various forms. One recognized form of sleep apnea is “central sleep apnea,” which is associated with a failure of the central nervous system to automatically initiate and control respiration. Another recognized form of sleep apnea is “obstructive sleep apnea,” which is associated with an obstruction of the airways due to airway collapse. Yet another recognized form of sleep apnea is a mixed form that may include a central nervous system failure to drive ventilatory effort combined with an obstructive apnea.

Possible effects of sleep apnea include daytime sleepiness, impaired alertness, and various associated cardiovascular diseases, which in turn can significantly impair patient lifestyle and increase morbidity risk. In some cases, obstructive sleep apnea can lead to death due to lack of oxygen to vital organs of the body. Various approaches have been taken to detect and treat sleep apnea.

### SUMMARY

A common way to detect sleep apnea has been with a sleep study. However, sleep studies need to be performed in a hospital setting which may involve long wait times and in which a patient may not sleep as well due to a different sleeping environment and/or discomfort due to placement of wires being used to detect sleep apnea, which affects the accuracy of the sleep apnea measurements. In addition, a sleep study can only reasonably be performed for a single night due to the costs and requirements of having a patient being in a hospital setting. Thus, sleep studies may provide less accurate sleep apnea episodes measurements and are unable to continuously and/or periodically monitor sleep apnea over a plurality of days.

In accordance with techniques described herein, an implantable medical device (IMD), such as an insertable cardiac monitor (ICM), is configured to continuously and/or periodically sense cardiac signals, such as electrocardiograms (ECGs), to detect one or more sleep apnea episodes of a patient occurring during a period of time based on the cardiac signal. For example, IMD may be configured to determine heart rates of the patient during the period of time and detect the one or more sleep apnea episodes of the patient occurring during the period of time based on the heart rates. The IMD is further configured to determine an amount of detected sleep apnea episodes of the patient during the period of time and compare the amount of detected sleep apnea episodes of the patient during the period of time to a sleep apnea severity threshold. The IMD is further configured to determine a classification of a sleep apnea burden of the patient based on the comparison. In some examples, the IMD is configured to determine a sleep apnea of burden of the patient for a plurality of consecutive days. The IMD being configured to determine sleep apnea burden classifications in accordance with techniques described herein that use an IMD that is particularly configured to sense a cardiac signal may provide improvements to the diagnostic capabilities of devices that detect sleep apnea episodes or sleep apnea burden by more accurately identifying and classifying a sleep apnea burden to a particular patient in which they are sleeping in a normal sleeping environment over a plurality of days. In some examples, an IMD described in techniques herein may provide a personalized determination of sleep apnea burden that adjusts for a particular patient that leads to more accurate determinations of classifications of sleep apnea burden for patient.

The IMD is configured to output one or more sleep apnea burden classifications to be used as an input to determine at least one of a heart failure (HF) risk score, sudden cardiac arrest (SCA) risk score, atrial fibrillation (AF) risk score, arrhythmia risk score, or chronic obstructive pulmonary disease (COPD) risk score, which may lead to more accurate determinations and/or predictions of those particular risk scores which provides an improvement to the diagnostic capabilities of the medical system including an IMD particularly configured to sense cardiac signals. In some examples, the respective risk scores may be determined by a device external to the IMD, such as a patient computing device, an edge computing device or a server.

Additionally, the techniques and systems of this disclosure may be implemented in an implantable medical device (IMD) that can continuously and/or periodically sense a cardiac signal without human intervention while subcutaneously implanted in a patient over days, months or years and perform millions of operations per second on patient cardiac signal data to detect sleep apnea episodes. Using techniques of this disclosure in an IMD may be advantageous when a physician cannot be continuously present with the patient over weeks or months to evaluate cardiac signal data while patient is sleeping and/or where performing millions of operations on weeks or months of cardiac signal data could not practically be performed in the mind of a physician with the techniques of this disclosure.

In one example, this disclosure describes a medical device comprising: sensing circuitry configured to sense a cardiac signal indicating activity of a heart of a patient; and processing circuitry configured to: detect one or more sleep apnea episodes of the patient occurring during a period of time based on the cardiac signal; compare an amount of detected sleep apnea episodes of the patient during the period of time to a sleep apnea severity threshold; and determine a classification of sleep apnea burden of the patient based on the comparison.

In another example, this disclosure describes a sensing, by sensing circuitry of a medical device, a cardiac signal indicating activity of a heart of a patient; detecting, by processing circuitry, one or more sleep apnea episodes of the patient occurring during a period of time based on the cardiac signal; comparing, by the processing circuitry, an amount of detected sleep apnea episodes of the patient during the period of time to a sleep apnea severity threshold; and determining, by the processing circuitry, a classification of sleep apnea burden during the period of time based on the comparison.

The details of one or more examples of this disclosure are set forth in the accompanying drawings and in the description below. Other features, objects, and advantages will be apparent from the description and drawings, and from the claims.

### BRIEF DESCRIPTION OF DRAWINGS

FIG. 1 is a conceptual diagram illustrating an example medical device system configured to detect sleep apnea in accordance with techniques of this disclosure.

FIG. 2 is a conceptual drawing illustrating an example configuration of a leadless implantable medical device of a medical device system.

FIG. 3 is a conceptual diagram illustrating another example medical device system configured to detect sleep apnea episodes in accordance with techniques of this disclosure.

FIG. 4 is a block diagram illustrating an example medical device configured to sense cardiac signals of a patient for detection of sleep apnea episodes and determination of sleep apnea burden classification in accordance with techniques of this disclosure.

FIG. 5 is a graph that illustrates sleep disordered heart rate (SDHR) information for a patient.

FIG. 6 is a flowchart illustrating a method by which a medical device system that is configured to detect a sleep apnea episode in accordance with techniques of this disclosure.

FIG. 7 is a flowchart illustrating a method by which a medical device system that is configured to monitor patient health based on sleep apnea episode detection in accordance with techniques of this disclosure.

### DETAILED DESCRIPTION

In general, this disclosure is directed to detecting episodes of sleep apnea over time, and determining and classifying sleep apnea burden. As used herein, “detection” refers to techniques for identifying an episode and does not necessarily imply that an episode has been identified. Accordingly, it is possible for a medical device system to initiate detection of sleep apnea episodes for a period of time and not detect any sleep apnea episodes (e.g., because no sleep apnea episodes occurred during the period of time).

Sleep apnea is a breathing disorder that cuts oxygen supply to various systems and organs of the body. To deal with the reduction in oxygenation levels, organs and systems of the body may trigger one or more compensatory mechanisms. With respect to the cardiovascular system, the compensatory mechanism(s) cause the heart to increase blood output for a period of time. As such, the cardiac compensatory mechanisms cause increased exertion of the heart. Moreover, at the end of a sleep apnea episode and during a recovery period that follows a sleep apnea episode, the patient’s heart rate may increase significantly, due to alveolar hyperventilation caused by the pulmonary system’s compensatory mechanisms. The heart rate spike after a sleep apnea episode may be greater in magnitude naturally occurring heart rate increases that are exhibited by the normal phenomenon of cyclical variation of heart rate (CVHR). As such, both the reduced oxygen supply during a sleep apnea episode and the hyperventilation that follows a sleep apnea episode may cause exertion levels in the heart that exceed normal levels of heart exertion.

The abnormal oxygenation conditions associated with sleep apnea may affect various systems and vital organs adversely. Repeated instances of increased heart exertion, as may be caused by frequent compensatory blood output to counter chronic sleep apnea and by increasing the heart rate to accommodate subsequent hyperventilation, increases the likelihood of heart ailments or possible heart failure. Some existing medical device systems have relied on respiratory measurement data to detect an episode of sleep apnea.

A medical device system may use heart rate to detect sleep apnea episodes of a patient. For example, the medical device system may detect sleep apnea episodes based on one or more heart rate parameters. In some examples, heart rate parameters may include heart rate variability (HRV) values, duration of a heart rate variation, magnitude of heart rate change, consecutiveness of apnea episodes, and other parameters that may be derived from HRV.

In accordance with techniques of this disclosure, a medical device system may use cardiac signals to continuously detect sleep apnea episodes during a sleeping event over a plurality of days and determine a sleep apnea burden based on the amount of sleep apnea episodes detected over a period of time during the sleeping event. The medical device system that includes an implantable medical device configured to detect sleep apnea episodes based on a sensed cardiac signal is able to determine a sleep apnea burden for each of a plurality of consecutive days, such as 5 consecutive days, 10 consecutive days, 30 consecutive days, etc. In some examples, a medical device system that determines a sleep apnea burden based an amount of detected sleep apnea episodes during a period of time and determines a sleep apnea burden for a plurality of days may input the determined sleep apnea burdens, such as for a plurality of consecutive days, as an input to determine at least one of a heart failure (HF) risk score, sudden cardiac arrest (SCA) risk score, atrial fibrillation (AF) risk score, arrhythmia risk score, or chronic obstructive pulmonary disease (COPD) risk score, which may lead to more accurate determinations and/or predictions of those particular risk scores which provides an improvement to the diagnostic capabilities of the medical system including an IMD particularly configured to sense cardiac signals. In some examples, the determination of respective risk scores may be determined by a device external to the IMD, such as a patient computing device, an edge computing device or a server.

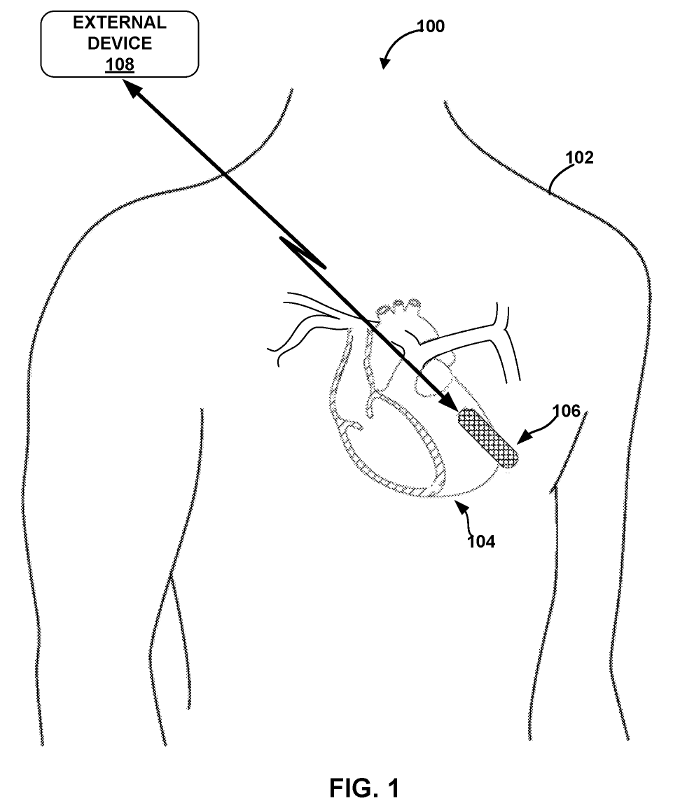

FIG. 1 is a conceptual diagram illustrating a medical device system 100 in conjunction with a patient 102. Medical device system 100 is an example of medical device system configured to implement the techniques described herein for detecting one or more sleep apnea episodes during a period of time and determining a classification of a sleep apnea burden during the period of time based on comparing an amount of detected sleep apnea episodes during the period of time to a sleep apnea severity threshold. In the example of FIG. 1, medical device system 100 may include a medical device 106 and an external device 108. Medical device 106 may be an implantable medical device (IMD), such as an insertable cardiac monitor (ICM), an implantable pacemaker, implantable defibrillator, implantable neurostimulator, etc. In some examples, although described primarily as implantable, medical device 106 is an external medical device, such as a patch, watch, headband, or other wearable medical device.

Medical device 106 is capable of sensing and recording cardiac electrogram (EGM) or electrocardiogram (ECG) signals from a position outside of heart 104 via electrodes of the medical device. In some examples, medical device 106 may include or be coupled to one or more additional sensors and/or sense one or more other physiological signals, such as signals that vary based on heart motion and/or sounds, blood pressure, blood flow, blood oxygenation, or respiration. For example, medical device 106 may be capable of measuring impedance via the electrodes, and generating an impedance signal that varies based on respiration based on the measured impedances. Medical device 106 may be implanted outside of the thorax of patient, e.g., subcutaneously or submuscularly, such as the pectoral location. In some examples, medical device 106 may take the form of a Reveal LINQ™ ICM.

External device 108 may be a computing device, e.g., used in a home, ambulatory, clinic, or hospital setting, to communicate with medical device 106 via wireless telemetry. External device 108 may be coupled to a remote patient monitoring system, such as Carelink^{®}. External device 108 may be, for example, a programmer, external monitor, or consumer device (e.g., smart phone). External device 108 may be used to program commands or operating parameters into medical device 106 for controlling its functioning, e.g., when configured as a programmer for medical device 106. External device 108 may be used to interrogate medical device 106 to retrieve data, including device operational data as well as physiological data accumulated in memory, such as quantifications of detected sleep apnea episodes and determinations of classification of a sleep apnea burden based on detected sleep apnea episodes during a period of time. The interrogation may be automatic, e.g., according to a schedule, or in response to a remote or local user command. Examples of communication techniques used by medical device 106 and external device 108 may include tissue conductance communication (TCC), or radiofrequency (RF) telemetry, which may be an RF link established via Bluetooth®, WiFi®, or medical implant communication service (MICS).

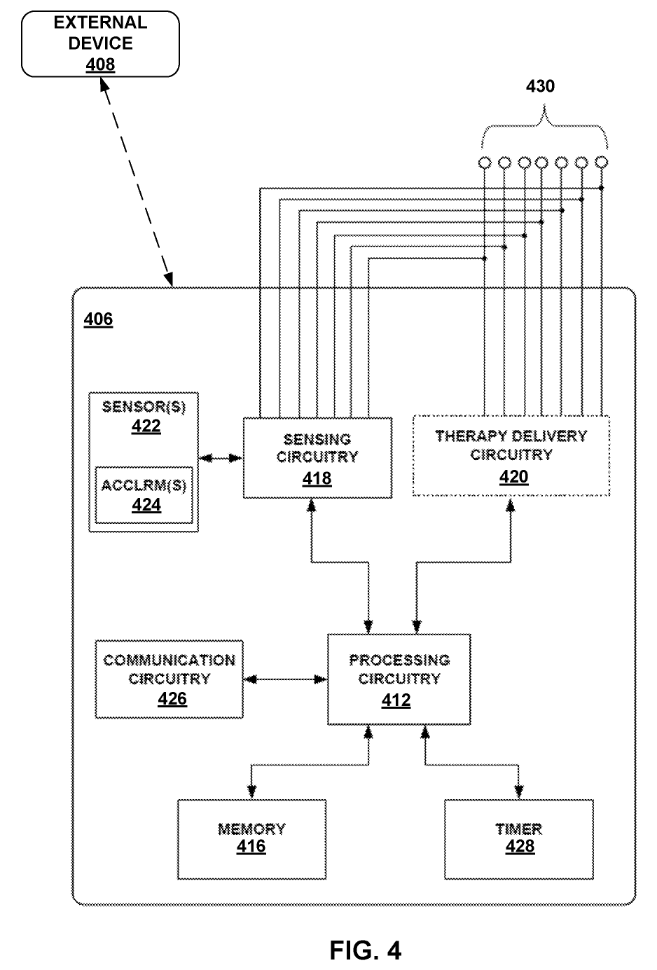

Both medical device 106 and external device 108 may include processing circuitry, and the processing circuitry of either device, of both devices, or any other device included in medical device system 100 may perform the techniques described herein. Based on analysis of patient’s heart activity in accordance with techniques of this disclosure, the processing circuitry of one or more of the devices (e.g., medical device 106 and/or external device 108 may also be configured to provide an indication to a user, e.g., to a clinician and/or to patient 102 of a classification of a sleep apnea burden during a period of time, such as during a sleeping period, e.g., via external device 108. For example, medical device system 100 may store (e.g., record) data associated with the occurrence of the detected sleep apnea episodes and the classification of the sleep apnea burden in memory (e.g., as illustrated in FIG. 4) included in medical device system 100 (e.g., memory of either medical device 106 or external device 108, or any other memory included in medical device system 100, such as memory included in a server, etc.). In some examples, medical device system 100 may then provide the determined sleep apnea burden classification as an input to determine at least one of a heart failure (HF) risk score, sudden cardiac arrest (SCA) risk score, atrial fibrillation (AF) risk score, arrhythmia risk score, or chronic obstructive pulmonary disease (COPD) risk score.

In some examples, medical device system 100 may present the data (e.g., as part of a report or history of patient 102) to patient 102, a clinician or caregiver of patient 102, or other user. Patient 102, a clinician, a caregiver or another implanted or external medical device may use the data to deliver or take a preventative measure to prevent the occurrence of additional sleep apnea episodes.

Although not illustrated in the example of FIG. 1, a medical device system configured to implement the techniques described in this disclosure may include one or more implanted or external medical devices in addition to or instead of medical device 106. For example, a medical device system may include a vascular implantable cardiac defibrillator (ICD), an extravascular ICD, a cardiac pacemaker implanted outside of the heart 104 but coupled to intracardiac or epicardial leads, or an intracardiac pacing device. One or more such devices may generate signals, and include processing circuitry configured to perform, in whole or in part, the techniques described herein for detecting sleep apnea episode(s). The implanted devices may communicate with each other and/or an external device 108, and one of the implanted or external devices may ultimately determine whether sleep apnea is detected based on information received from the other device(s).

In various examples, implantable medical device (IMD) components may be connected to leads that extend into heart 104 or could be implanted in heart 104 entirely. In some examples, components of medical device system 100 may be external devices. Components of medical device system 100 may be configured to detect cardiac electrogram signals, such as an ECG. In various examples, processing circuitry of medical device system 100, such as processing circuitry of medical device 106 and/or external device 108, may perform the techniques of this disclosure using various types of sensing circuitry, such as sensing circuitry of medical device 106. For instance, the sensing circuitry may be configured to sense a cardiac signal indicating activity of heart 104 of patient 102. Medical device 106 may include electrodes configured to sense electrical signals associated with patient state. The electrodes may be coupled to the sensing circuitry. In some examples, a cardiac signal may include an ECG signal, an EGM signal, a heart sound signal or any other sensed signal indicative of activity of the heart.

Processing circuitry of medical device system 100 may be configured to determine heart rates of patient 102 based on a cardiac signal sensed during a period of time. The processing circuitry may use heart rate (described in greater detail below) to the determined heart rates to detect one or more sleep apnea episodes of the patient occurring during the period. In some examples, the processing circuitry may determine one or more heart rate parameters (discussed in greater detail below) based on the determined heart rates of patient 102. The processing circuitry may detect sleep apnea episodes of patient 102 occurring during first period based on the one or more heart rate parameters.

It should be understood that in some examples the processing circuitry may detect sleep apnea episodes occurring during the period of time after the period of time. That is, in some examples, sensing circuitry may sense the cardiac signal during the period of time, and the processing circuitry may analyze the cardiac signal for the period of time after the period of time to detect whether one or more sleep apnea episodes occurred during the period of time. In some examples, the period of time includes a sleeping event, such as a nighttime sleeping event. In some examples, a duration of the period of time may be for at least 1 hour, be for at least 3 hours, be for at least 6 hours. In some examples, there may be a plurality of periods of time during one sleeping event, such as a plurality of time periods, such as a plurality of 1-hour periods of time or other periods of time with either the same or varying durations.

The processing circuitry may be configured to determine whether one or more sleep apnea severity thresholds are satisfied. For example, processing circuitry may determine an amount detected sleep apnea episodes during a period of time and compare the amount of detected sleep apnea episodes of the patient 102 to a sleep apnea severity threshold and determine a classification of sleep apnea burden during the period of time based on the comparison. In some examples, a sleep apnea severity threshold may include a plurality of threshold levels, such as “mild”, “moderate”, and “severe.” In some examples, a severe sleep apnea burden threshold may be at least 30 detected sleep apnea episodes during the period of time. In some examples, a severe sleep apnea burden threshold may be at least 30 detected sleep apnea episodes during the period of time. In some examples, a mild sleep apnea burden threshold may be 15 or less detected sleep apnea episodes during the period of time. For example, processing circuitry may determine the classification of the detected sleep apnea episodes is severe based on the amount of detected sleep apnea episodes satisfying the severe sleep apnea burden threshold (e.g., at least 30 sleep apnea episodes during the period of time) or may determine the classification of the detected sleep apnea episodes is mild based on the amount of detected sleep apnea episodes satisfying the mild sleep apnea burden threshold (e.g., detecting 15 or less sleep apnea episodes during the period of time). In some examples, the amount of sleep apnea during the period of time may be a cumulative duration of one or more episodes detected during the period of time, and the threshold may be a duration threshold, e.g., number of minutes.

In some examples, the amount of detected sleep apnea episodes during a period of time may correspond to a rate of detected sleep apnea episodes during the sleeping event. For example, an average number of detected sleep apnea episodes per period of time, such as an hour. In some examples, a sleep apnea severity threshold may include a sleep apnea severity rate threshold. In some examples, processing circuitry determining the detected sleep apnea episodes satisfy a severe sleep apnea burden threshold may include detecting at least 30 sleep apnea episodes per hour during a sleeping event. In some examples, processing circuitry determining the detected sleep apnea episodes satisfy a severe sleep apnea burden threshold may include detecting at least 35 sleep apnea episodes per hour during a sleeping event. In some examples, processing circuitry determining the detected sleep apnea episodes satisfy a mild sleep apnea burden threshold may include detecting 5 or less sleep apnea episodes per hour during a sleeping event. In some examples, processing circuitry may determine a sleep apnea burden is moderate when the amount of sleep apnea episodes occur between 15 and 30 times during the period of time. In some examples, the amount of detected sleep apnea episodes during the period may be expressed as a fraction, percentage, or other portion of time during which patient 102 was experiencing sleep apnea.

In some examples, processing circuitry may be configured to determine at least one of a magnitude of heart rate change or a duration of heart rate change based on the determined heart rates sensed during a period of time. In some examples, processing circuitry may be configured to classify at least one sleep apnea episode of the one or more sleep apnea episodes as obstructive sleep apnea or central sleep apnea based on at least one of the magnitude of heart rate change or the duration of heart rate change. For example, a magnitude of heart rate change in a sleep apnea episode may be greater for obstructive sleep apnea than for central sleep apnea. In some examples, processing circuitry may determine a sleep apnea episode is an obstructive sleep apnea episode based on the magnitude of heart change satisfying an obstructive sleep apnea threshold (e.g. when the magnitude of heart rate change is above the obstructive sleep apnea threshold, the sleep apnea episode is classified as an obstructive sleep apnea episode).

In some examples, a duration of heart change during a sleep apnea episode may indicate whether a sleep apnea episode is an obstructive sleep apnea episode or a central sleep apnea episode. For example, a duration of heart change during the sleep apnea episode may be shorter for obstructive sleep apnea when compared to central sleep apnea. In some examples, processing circuitry may determine a sleep apnea episode is an obstructive sleep apnea episode based on the duration of heart change during a sleep apnea episode satisfying a duration obstructive sleep apnea threshold (e.g. when the duration of heart rate change is below the obstructive sleep apnea threshold, the sleep apnea episode is classified as an obstructive sleep apnea episode).

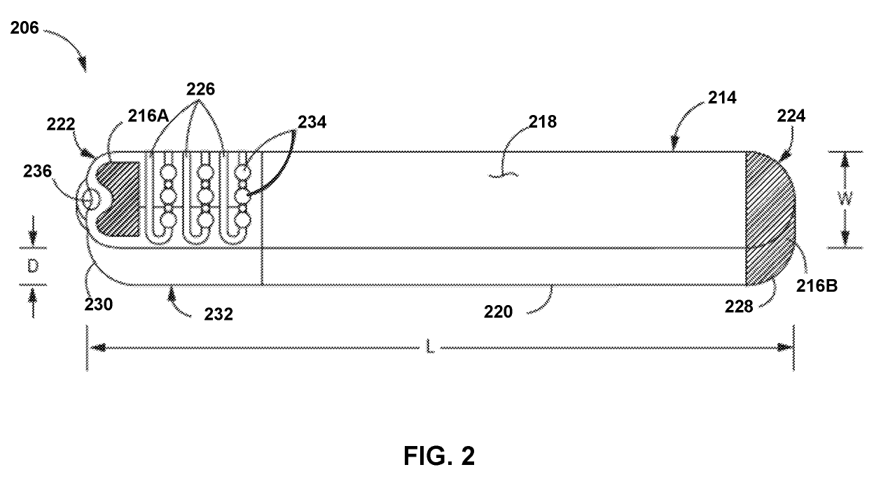

FIG. 2 is a conceptual drawing illustrating an example configuration of an IMD 206. IMD 206 may be substantially similar to medical 106 of FIG. 1. In the example shown in FIG. 2, IMD 206 may comprise a leadless, subcutaneously-implantable monitoring device having a housing 214, a proximal electrode 216A, and a distal electrode 216B. Housing 214 encloses electronic circuitry located inside the IMD 206, and protects the circuitry contained therein from fluids such as body fluids. In some examples, housing 214 may comprise first major surface 218, second major surface 220, proximal end 222, and distal end 224. Proximal electrode 216A and distal electrode 216B may be positioned near respective proximal and distal ends 222 and 224 of IMD 206, such that a spacing between proximal electrode 216A and distal electrode 216B may range from about 30–55 mm, about 35–55 mm, or about 40–55 mm, or more generally from about 25–60 mm. In some examples, IMD 206 may include one or more additional electrodes and/or one or more other sensors (not shown), which may be positioned on one or both of major surfaces 218, 220 of IMD 206. In any such examples, electrical feedthroughs may provide electrical connection of electrodes 216A, 216B or other sensors to circuitry within housing 214.

In the example shown in FIG. 2, IMD 206 is defined by a length L, a width W, and thickness or depth D. In this example, IMD 206 is in the form of an elongated rectangular prism in which length L is significantly greater than width W, and in which width W is greater than depth D. However, other configurations of IMD 206 are contemplated, such as those in which the relative proportions of length L, width W, and depth D vary from those described and shown in FIG. 2. In some examples, the geometry of the IMD 206, such as the width W being greater than the depth D, may be selected to allow IMD 206 to be inserted under the skin of the patient using a minimally invasive procedure and to remain in the desired orientation during insertion. In addition, IMD 206 may include radial asymmetries (e.g., the rectangular shape) along a longitudinal axis of IMD 206, which may help maintain the device in a desired orientation following implantation.

Overall, IMD 206 may have a length L of about 20–30 mm, about 40–60 mm, or about 45–60 mm. In some examples, the width W of first major surface 218 may range from about 3–10 mm, and may be any single width or range of widths between about 3–10 mm. In some examples, a depth D of IMD 206 may range from about 2–9 mm. In other examples, the depth D of IMD 206 may range from about 2–5 mm, and may be any single or range of depths from about 2–9 mm. In any such examples, IMD 206 is sufficiently compact to be implanted within the subcutaneous space of patient 102 in the region of a pectoral muscle.

IMD 206 may have a geometry and size designed for ease of implantation and patient comfort. For example, IMD 206 may have a volume of 3 cubic centimeters (cm³) or less, 1.5 cm³ or less, or any volume therebetween. As illustrated in FIG. 2, proximal end 222 and distal end 224 may be rounded, which may reduce discomfort and/or irritation to surrounding tissue when IMD 206 implanted under the skin of patient 102. An example configuration of IMD 206, including an instrument and method for inserting IMD 206 is described in U.S. Patent Publication No. 2014/0276928. An example configuration of IMD 206 also is described in U.S. Patent Publication No. 2016/0310031.

In some examples, IMD 206 may be configured for implantation within patient 102 such that first major surface 218 of IMD 206 faces outward towards the skin when IMD 206 is inserted within patient 102 and second major surface 220 is faces inward toward musculature of patient 102. First and second major surfaces 218, 220 may face in directions along a sagittal axis of patient 102, as illustrated in FIG. 1, and this orientation may be maintained upon implantation due to the dimensions of IMD 206. Additionally, or alternatively, IMD 206 may be configured for implantation within patient 102 in one or more other orientations relative to one or more anatomical landmarks of patient 102.

In the example shown in FIG. 2, proximal end 222 of IMD 206 includes header assembly 232 having one or more of integrated antenna 226, anti-migration projections 234, and suture hole 236. Integrated antenna 226 is located on the same major surface (e.g., first major surface 218) as electrode 216A, and may be an integral part of header assembly 232. In other examples, integrated antenna 226 may be formed on the major surface opposite from electrode 216A or may be incorporated within housing 214 of IMD 206. Antenna 226 may be configured to transmit or receive electromagnetic signals for communication. For example, antenna 226 may be configured to transmit to or receive signals from a programmer via inductive coupling, electromagnetic coupling, tissue conductance, Near Field Communication (NFC), Radio Frequency Identification (RFID), Bluetooth, WiFi, or other proprietary or non-proprietary wireless telemetry communication schemes. Antenna 226 may be coupled to communication circuitry of IMD 206, which may drive antenna 226 to transmit signals to an external device (e.g., external device 108). Antenna 226 may transmit signals received from the external device to processing circuitry of IMD 206 via the communication circuitry.

IMD 206 may include several features for retaining IMD 206 in position once subcutaneously implanted in patient 102. For example, as shown in FIG. 2, housing 214 may include anti-migration projections 234 positioned adjacent integrated antenna 226. Anti-migration projections 234 may comprise a plurality of bumps or protrusions extending away from first major surface 218 and may reduce or prevent movement of IMD 206 after implantation in patient 102. In other examples, anti-migration projections 234 may be located on the opposite major surface as proximal electrode 216A and/or integrated antenna 226. In addition to or instead of anti-migration projections 234, a portion of housing 214 (e.g., header assembly 232) may define a suture hole 236, which may enable a clinician to suture IMD 206 to patient tissue to reduce or prevent movement of IMD 206 after implantation in patient 102. In the example of FIG. 2, suture hole 236 is defined by a portion of header assembly 232 adjacent to proximal electrode 216A. In some examples, header assembly 232 may comprise a molded header assembly made from a polymeric material, which may be integral with or separable from the main portion of IMD 206.

In examples in which the processing circuitry of the medical device system including IMD 206 is configured to measure heart activity based on a cardiac electrogram signal, IMD 206 may include a plurality of electrodes. For example, as illustrated in FIG. 2, IMD 206 may include a proximal electrode 216A and a distal electrode 216B. As shown in the illustrated example, proximal electrode 216A may be positioned on header assembly 232, and distal electrode 216B may be formed from an uninsulated distal portion of conductive housing 214. Proximal electrode 216A and distal electrode 216B may be positioned near respective proximal and distal ends 222 and 224 of IMD 10, such that a spacing between proximal electrode 216A and distal electrode 216B may range from about 30–55 mm, about 35–55 mm, or about 40–55 mm, or more generally from about 25–60 mm. In some examples, IMD 206 also may include one or more additional electrodes (not shown) positioned on one or both of major surfaces 218, 220 of IMD 10. In any such examples, electrical feedthroughs may provide electrical connection of electrodes 216A, 216B, any additional electrodes, and antenna 226, to circuitry within housing 214.

In the example shown in FIG. 2, proximal electrode 216A is in close proximity to proximal end 222, and distal electrode 216B is in close proximity to distal end 224 of IMD 10. In this example, distal electrode 216B is not limited to a flattened, outward facing surface, but may extend from first major surface 218, around rounded edges 228 or end surface 230, and onto the second major surface 220 in a three-dimensional curved configuration. As illustrated, proximal electrode 216A is located on first major surface 218 and is substantially flat and outward facing. However, in other examples not shown here, proximal electrode 216A and distal electrode 216B both may be configured like proximal electrode 216A shown in FIG. 2, or both may be configured like distal electrode 216B shown in FIG. 2. Any of electrodes 216A, 216B may be formed of a biocompatible conductive material. For example, any of electrodes 216A, 216B may be formed from any of stainless steel, titanium, platinum, iridium, or alloys thereof. In addition, electrodes of IMD 206 may be coated with a material such as titanium nitride or fractal titanium nitride, although other suitable materials and coatings for such electrodes may be used.

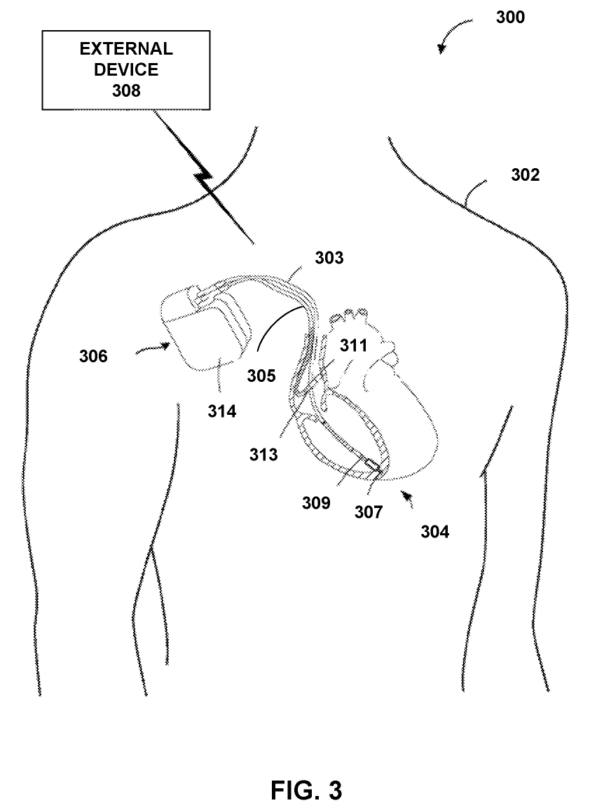

FIG. 3 is a conceptual drawing illustrating an example medical device system 300 in conjunction with a patient 302. Medical device system 300 may be substantially similar to medical device system 100 except for any differences described herein. Medical device system 100 and medical device system 300 are examples of medical device systems configured to implement the techniques described herein for detecting a sleep apnea episode. In the illustrated example, medical device system 300 includes a medical device 306 coupled to a ventricular lead 303 and an atrial lead 305. In some examples, upon identifying a likely sleep apnea episode, medical device system 300 may responsively provide an indication indicating that a sleep apnea episode is likely occurring, and/or deliver a therapy (e.g., using ventricular lead 303 and atrial lead 305) configured to prevent, mitigate, or remedy the effects of the sleep apnea episode.

Ventricular lead 303 and atrial lead 305 may be electrically coupled to medical device 306 and extend into the patient's heart 304. Ventricular lead 303 may include electrodes 307 and 309 shown positioned on the lead in the patient's right ventricle (RV) for sensing EGM signals and pacing in the RV. Atrial lead 305 may include electrodes 311 and 313 positioned on the lead in the patient's right atrium (RA) for sensing atrial EGM signals and pacing in the RA.

Medical device 306 may use both ventricular lead 303 and atrial lead 305 to acquire cardiac electrogram (EGM) signals from heart 304 of patient 102B. Medical device system 300 is shown as having a dual chamber IMD configuration, but other examples may include one or more additional leads, such as a coronary sinus lead extending into the right atrium, through the coronary sinus and into a cardiac vein to position electrodes along the left ventricle (LV) for sensing LV EGM signals and delivering pacing pulses to the LV. In other examples, a medical device system may be a single chamber system, or otherwise not include atrial lead 305.

Processing circuitry, sensing circuitry, and other circuitry configured for performing the techniques described herein may be housed within a sealed housing 314 of medical device 306. Housing 314 (or a portion thereof) may be conductive to serve as an electrode for pacing or sensing. Medical device 306 may acquire signal data (e.g., EGM signal data) and cardiac rhythm episode data and transmit the data to an external device 308. External device 308 may be a computing device, e.g., a device used in a home, ambulatory, clinic, or hospital setting, comprising processing circuitry and/or communicative interfacing circuitry configured to communicate with medical device 306 via wireless telemetry. External device 308 may be coupled to a remote patient monitoring system, such as Carelink^{®}. External device 308 may include, be, or may be part of, in various examples, a programmer, external monitor, or consumer device, e.g., a smart phone.

External device 308 may be implemented and may operate in a manner similar to external device 108. For example, external device 308 may be used to program commands or operating parameters into medical device 306 for controlling its functioning, e.g., when configured as a programmer for medical device 306, and may be used to interrogate medical device 306 to retrieve data, including device operational data as well as physiological data accumulated in a memory of medical device 306. Medical device system 300 is an example of a medical device system operable to leverage physiological parameter value(s) to detect sleep apnea episodes. For example, medical device system 300 may be configured to monitor physiological parameter value(s) and determine whether one or more physiological parameter value satisfy one or more conditions. In some examples, if medical device system 300 determines that a sleep apnea episode has likely occurred, medical device system 300 may responsively provide an indication indicating that the event is detected, and optionally, trigger delivery of a therapy configured to remediate the effects of the event or stem the progression of such effects. The techniques may be performed by processing circuitry of medical device system 300, such as processing circuitry of one or both of medical device 306 and external device 308, individually, or collectively.

The processing circuitry of external device 308 and/or medical device 306 may determine the values of at least some patient parameters based on signals generated by sensing circuitry of medical device 306. In some examples, medical device 306 may include or be coupled to one or more other sensors that generate one or more other physiological signals, such as signals that vary based on blood flow and/or respiration. The processing circuitry of external device 308 and/or medical device 306 may determine patient parameters based on therapy delivered by various components of medical device system 300 that are omitted from FIG. 3 for ease of illustration, such as a CPAP machine. For instance, the processing circuitry of external device 308 and/or medical device 306 may analyze physiological parameters of patient 302 to determine whether a sleep apnea episode has been remediated effectively by way of therapy delivered by the positive airway pressure machine or medical device 306. In some examples, a user, such as patient 302, may provide a user input that indicates whether a CPAP machine was used during a period of time, such as during a sleeping event.

FIG. 4 is a functional block diagram illustrating an example configuration of a medical device 406. Medical device 406 may correspond to medical device 106 in FIG. 1, IMD 206 in FIG. 2, medical device 306 in FIG. 3, or another medical device configured to implement the techniques as described in this disclosure. Similarly, external device 408 may correspond to external device 108 in FIG. 1, external device 308 illustrated in FIG. 3, or another external device configured to implement the techniques as described in this disclosure.

In the illustrated example, medical device 406 includes processing circuitry 412, memory 416, sensing circuitry 418, therapy delivery circuitry 420, one or more sensors 422 (e.g., an accelerometer 424), communication circuitry 426, and timer 428. However, medical device 406 does not need to include all these components in some examples, or medical device 406 may include additional components in some examples. For example, medical device 406 may not include therapy delivery circuitry 420, in some examples.

Memory 416 may include computer-readable instructions that, when executed by processing circuitry 412, cause medical device 406 and processing circuitry 412 to perform various functions attributed to medical device 406 and processing circuitry 412 herein (e.g., determining time of day, comparing time of day to a predetermined window, causing communication circuitry 426 to transmit physiological parameter value(s) to external device, etc.). Memory 416 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital or analog media. Memory 416 may store threshold(s) for the peak-to-valley time interval condition, the activity count condition, the peak-to-valley heart rate variation condition, the cycle length condition, sleep apnea burden severity, etc.

Processing circuitry 412 may include fixed function circuitry and/or programmable processing circuitry. Processing circuitry 412 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 412 may include multiple components, such as any combination of one or more microprocessors, one or more controllers, one or more DSPs, one or more ASICs, or one or more FPGAs, as well as other discrete or integrated logic circuitry. The functions attributed to processing circuitry 412 herein may be embodied as software, firmware, hardware or any combination thereof. For example, processing circuitry 412 may be processing circuitry of either medical device 406 or external device 408 or any other processing circuitry included in a medical device system may be configured to perform techniques in accordance with this disclosure.

Sensing circuitry 418 and therapy delivery circuitry 420 are coupled to electrodes 430. Electrodes 430 illustrated in FIG. 4 may correspond to, for example, electrodes carried on leads 303 and/or 305 of medical device system 300 (FIG. 3). Sensing circuitry 418 may monitor signals from a selected two or more of electrodes 430 in order to monitor electrical activity of heart, impedance, or other electrical phenomenon. For example, sensing circuitry 418 may be configured to, via electrodes 430, sense a cardiac signal indicating activity of heart 104 of patient 102, measure impedances of patient 102, etc. In some examples, sensing circuitry 418 may include one or more filters and amplifiers for filtering and amplifying a signal received from electrodes 430.

Sensing circuitry 418 may include a switch module to select which of the available electrodes 430 (or electrode polarities) are used to sense the heart activity. In examples with several electrodes 430, processing circuitry 412 may select the electrodes that function as sense electrodes, i.e., select the sensing configuration, via the switch module within sensing circuitry 418. Sensing circuitry 418 may also pass one or more digitized cardiac signals, such as an EGM or ECG signal, to processing circuitry 412 for analysis, e.g., for use in cardiac activity discrimination (e.g., cardiac rhythm discrimination).

In the example of FIG. 4, medical device 406 includes one or more sensors 422 coupled to sensing circuitry 418. Although illustrated in FIG. 4 as being included within medical device 406, one or more of sensors 422 may be external to medical device 406, e.g., coupled to medical device 406 via one or more leads, or configured to wirelessly communicate with medical device 406. In some examples, sensors 422 transduce a signal indicative of a patient parameter, which may be amplified, filtered, or otherwise processed by sensing circuitry 418. In such examples, processing circuitry 412 determines physiological parameter value(s) based on the signals. In some examples, sensors 422 determine the physiological parameter value(s), and communicates them, e.g., via a wired or wireless connection, to processing circuitry.

In some examples, sensors 422 may include one or more accelerometers 424, e.g., one or more three-axis accelerometers. Signals generated by the one or more accelerometers 424 may be indicative of, for example, heart sounds or other vibrations or movement associated with the beating of the heart, or coughing, rales, or other respiration abnormalities. Accelerometers 424 may produce and transmit signals to processing circuitry 412 for a determination as to whether the patient’s heart has contracted. In some examples, sensors 422 may include one or more microphones configured to detect heart sounds or respiration abnormalities. In some examples, sensors 422 may include sensors configured to transduce signals indicative of blood flow, oxygen saturation of blood, or patient temperature, and processing circuitry may determine patient parameters values based on these signals. In some examples, sensors 422 may include optical sensors configured to detect optical signals, such as a photoplethysmography (PPG) signal. In some examples, sensors may include optical sensors configured to detect optical signals that indicate at least one of an amount or level of tissue oxygenation and/or an amount or level of blood oxygen.

Therapy delivery circuitry 420 is configured to generate and deliver electrical therapy to the heart. Therapy delivery circuitry 420 may include one or more pulse generators, capacitors, and/or other components capable of generating and/or storing energy to deliver as pacing therapy, defibrillation therapy, cardioversion therapy, other therapy or a combination of therapies. In some instances, therapy delivery circuitry 420 may include a first set of components configured to provide pacing therapy and a second set of components configured to provide anti-tachyarrhythmia shock therapy. In other instances, therapy delivery circuitry 420 may utilize the same set of components to provide both pacing and anti-tachyarrhythmia shock therapy. In still other instances, therapy delivery circuitry 420 may share some of the pacing and shock therapy components while using other components solely for pacing or shock delivery.

Therapy delivery circuitry 420 may include charging circuitry, one or more charge storage devices, such as one or more capacitors, and switching circuitry that controls when the capacitor(s) are discharged to electrodes 430 and the widths of pulses. Charging of capacitors to a programmed pulse amplitude and discharging of the capacitors for a programmed pulse width may be performed by therapy delivery circuitry 420 according to control signals received from processing circuitry, which are provided by processing circuitry according to parameters stored in memory 416. Processing circuitry 412 may control therapy delivery circuitry 420 to deliver the generated therapy to the heart via one or more combinations of electrodes 430, e.g., according to parameters stored in memory 416. Therapy delivery circuitry 420 may include switch circuitry to select which of the available electrodes 430 are used to deliver the therapy, e.g., as controlled by processing circuitry.

Communication circuitry 426 may include any suitable hardware, firmware, software or any combination thereof for communicating with another device, such as an external device 408 or another IMD or sensor. Under the control of processing circuitry, communication circuitry 426 may receive downlink telemetry from and send uplink telemetry to external device 408 or another device with the aid of an antenna, which may be internal and/or external. In some examples, communication circuitry 426 may communicate with a local external device, and processing circuitry may communicate with a networked computing device via the local external device and a computer network, such as the Medtronic CareLink® Network.

A clinician or other user may retrieve data from medical device 406 using external device 408 or another local or networked computing device configured to communicate with processing circuitry via communication circuitry 426. The clinician may also program parameters of medical device 406 using external device 408 or another local or networked computing device.

Although not illustrated in FIG. 4, communication circuitry 426 may be coupled to electrodes 430 for tissue conductance communication (TCC) via the electrodes. In some examples, communication with medical device 406 and external device 408 may be via RF telemetry or TCC.

Medical device 406 and/or external device 408 may include timer 428. Timer 428 may be configured to provide a timer value, which processing circuitry may use to measure and determine various physiological parameters that involve duration. Additionally or alternatively, processing circuitry 412 may determine a time of day based on the timer value. For example, processing circuitry 412 may be configured to determine a time of day based on the timer value. Processing circuitry 412 may associate heart activity of the patient with the time of day when the heart activity occurred. For example, processing circuitry 412 may associate one or more sleep apnea episodes with the time of day when the sleep apnea episodes occurred and store this information in memory 416, which may be retrieved, for example, during interrogation by external device 408.

Processing circuitry 412 may be configured to determine physiological parameters and/or conditions of patient 102. For example, processing circuitry 412 may be configured to determine heart rates of patient 102 based on a cardiac signal (e.g., obtained by sensing circuitry 418), detect sleep apnea episodes based on heart rate, detect atrial fibrillation episodes based on a cardiac signal, determine HRV values based on determined heart rates, determine whether a sleep apnea episodes is an obstructive sleep apnea episode or central sleep apnea episode based on at least one of a magnitude of heart rate change during a sleep apnea episodes or a duration of heart rate change during a sleep apnea episodes, determine a classification of a sleep apnea burden based on an amount of detected sleep apnea episodes during a period of time, etc. Processing circuitry 412 may use one or more algorithms or methods to determine HRV values. Example methods may include the “Root Mean Square of Successive Differences” (RMSSD), the standard deviation of N-N intervals (SDNN), the standard deviation of sequential 5-minute N-N intervals (SDANN).

In some examples, processing circuitry 412 may be configured to determine a sleep apnea burden based on one or more of determined physiological parameter value(s), frequency of detected sleep apnea episodes, classification of whether sleep apnea episodes are obstructive sleep apnea or central sleep apnea duration of a sleep apnea episode, change in sleep apnea burden over a period of time, such as a daily change in sleep apnea burden (e.g., day-to-day changes in sleep apnea burden over time), determinations of sleep apnea burden trends over time, whether patient 102 is non-complaint with continuous positive airway pressure (CPAP), heart rate variability of patient 102, presence of atrial fibrillation (AF) in patient 102, risk of heart failure, or risk of chronic obstructive pulmonary disease (COPD) exacerbation.

In some examples, processing circuitry 412 may be configured to detect one or more sleep apnea episodes of the patient occurring during a period of time based on a cardiac signal sensed during the period of time. In some examples, processing circuitry 412 may be configured to determine heart rates of patient 102 based on a cardiac signal sensed during a period of time. In some examples, processing circuitry 412 may be configured to use the determined heart rates to detect one or more sleep apnea episodes of the patient occurring during a period of time. In some examples, the processing circuitry may determine one or more heart rate parameters based on the determined heart rates of patient 102. The processing circuitry may detect sleep apnea episodes of patient 102 occurring during a period of time based on the one or more heart rate parameters.

In some examples, processing circuitry 412 may be able to determine an amount of sleep apnea episodes occurring during a period of time and/or determine a rate of sleep apnea episodes occurring during a period of time. In some examples, the period of time includes a sleeping event, such as a nighttime sleeping event. In some examples, a duration of the period of time may be for at least 1 hour, be for at least 3 hours, be for at least 6 hours. In some examples, there may be a plurality of periods of time during one sleeping event, such as a plurality of time periods, such as a plurality of 1-hour periods of time or other periods of time with either the same or varying durations.

Processing circuitry 412 may be configured to compare an amount of sleep apnea episodes detected during the period of time to a sleep apnea severity threshold and determine a classification of a sleep apnea burden during the period of time based on the comparison. For example, processing circuitry 412 may be configured to determine whether one or more sleep apnea severity thresholds are satisfied. In some examples, a sleep apnea severity threshold may include a plurality of threshold levels, such as “mild”, “moderate”, or “severe.” In some examples, a severe sleep apnea burden threshold may be at least 30 detected sleep apnea episodes during the period of time. In some examples, a severe sleep apnea burden threshold may be at least 35 detected sleep apnea episodes during the period of time. In some examples, a mild sleep apnea burden threshold may be 15 or less detected sleep apnea episodes during the period of time. For example, processing circuitry 412 may determine the classification of the detected sleep apnea episodes is severe based on the amount of detected sleep apnea episodes satisfying the severe sleep apnea burden threshold (e.g., at least 30 sleep apnea episodes during the period of time) or may determine the classification of the detected sleep apnea episodes is mild based on the amount of detected sleep apnea episodes satisfying the severe sleep apnea burden threshold (e.g., detecting 5 or less sleep apnea episodes during the period of time).

In some examples, the detected sleep apnea episodes during a period of time may correspond to a rate of detected sleep apnea episodes during the sleeping event. For example, the detected sleep apnea episodes during a period of time may correspond to a rate of detected sleep apnea episodes per period of time, such as an hour (e.g., at least 30 detected sleep apnea episodes per hour corresponds to a severe sleep apnea burden). In some examples, a sleep apnea severity threshold may include a sleep apnea severity rate threshold. In some examples, processing circuitry 412 determining the detected sleep apnea episodes satisfy a severe sleep apnea burden threshold may include detecting at least 30 sleep apnea episodes per hour during a sleeping event. In some examples, processing circuitry 412 determining the detected sleep apnea episodes satisfy a severe sleep apnea burden threshold may include detecting at least 35 sleep apnea episodes per hour during a sleeping event. In some examples, processing circuitry 412 determining the detected sleep apnea episodes satisfy a mild sleep apnea burden threshold may include detecting 15 or less sleep apnea episodes per hour during a sleeping event.

In some examples, processing circuitry 412 may determine a personalized sleep apnea burden threshold, such as a personalized severe sleep apnea burden threshold, based on historic sleep apnea trends of the patient. In some examples, processing circuitry 412 may be configured to store historic sleep apnea trends in memory 416. In some examples, historic sleep apnea trends may include at least one of data related to sleep apnea episodes of patient 102 sensed by medical device 406 and/or data related to sleep apnea episodes of patient 102 received from a device other than medical device 406, such as external device 108. For example, if patient has higher amounts of historic sleep apnea episodes, processing circuitry 412 may increase a sleep apnea burden threshold. For example, if patient has higher amounts of historic sleep apnea episodes, processing circuitry 412 may increase a severe sleep apnea burden threshold from a first threshold amount to a second threshold amount, such as from 30 to 35.

In some examples, processing circuitry 412 may be configured to determine at least one of a magnitude of heart rate change or a duration of heart rate change based on the determined heart rates sensed during a period of time. In some examples, processing circuitry 412 may be configured to classify at least one sleep apnea episode of the one or more sleep apnea episodes as obstructive sleep apnea or central sleep apnea based on at least one of the magnitude of heart rate change during the sleep apnea episode or the duration of heart rate change during the sleep apnea episodes. For example, a magnitude of heart rate change in a sleep apnea episode may be greater for obstructive sleep apnea than for central sleep apnea. In some examples, processing circuitry 412 may determine a sleep apnea episode is an obstructive sleep apnea episode based on the magnitude of heart change satisfying an obstructive sleep apnea threshold (e.g. when the magnitude of heart rate change is above the obstructive sleep apnea threshold, the sleep apnea episode is classified as an obstructive sleep apnea episode).

In some examples, a duration of heart change during a sleep apnea episode may indicate whether a sleep apnea episode is an obstructive sleep apnea episode or a central sleep apnea episode. For example, a duration of heart change during the sleep apnea episode may be shorter for obstructive sleep apnea when compared to central sleep apnea. In some examples, processing circuitry 412 may determine a sleep apnea episode is an obstructive sleep apnea episode based on the duration of heart change during a sleep apnea episode satisfying a duration obstructive sleep apnea threshold (e.g. when the duration of heart rate change is below the obstructive sleep apnea threshold, the sleep apnea episode is classified as an obstructive sleep apnea episode).

In some examples, processing circuitry 412 may be configured to determine whether patient was non-complaint with CPAP during the period of time and determine a classification of sleep apnea burden during the period of time based on the comparison of the amount of detected sleep apnea episodes of the patient during the period of time to a sleep apnea severity threshold and the determination of whether the patient was non-complaint with CPAP during the period of time. In some examples, in response to determining patient was non-complaint with CPAP during the period of time, processing circuitry 412 may increase the severity of sleep apnea burden (e.g., increasing a classification from moderate to severe). In some examples, in response to determining patient was complaint with CPAP during the period of time, processing circuitry 412 may decrease the severity of sleep apnea burden (e.g., decreasing a classification from moderate to mild).

In some examples, processing circuitry 412 may be configured to determine a change in sleep apnea burden over a period of time, such as day-to-day changes in sleep apnea burden. In some examples, processing circuitry 412 may be configured to determine a total sleep apnea burden based on a plurality of determined daily sleep apnea burdens, such as changes in daily sleep apnea burden values over time, such as over days, weeks, or even months. In some examples, more recent daily sleep apnea burden values (e.g., a sleep apnea burden determination of a day (e.g., a sleep apnea burden being classification as “severe”) may be weighted heavier than older daily sleep apnea burden values (e.g., a sleep apnea burden determination within 24 hours prior may be weighted greater than a month-old sleep apnea burden determination).

In some examples, processing circuitry 412 may be configured to determine at least one of a HF risk score, SCA risk score, arrhythmia risk score, or COPD risk score based, at least in part, on the classification of the sleep apnea burden. In some examples, processing circuitry 412 may be configured to determine at least one of a HF risk score, SCA risk score, arrhythmia risk score, or COPD risk score based, at least in part, on the classification of the sleep apnea burden, such as during a sleeping event, and at least one of the classification of the at least one sleep apnea episode of the one or more sleep apnea episodes as obstructive sleep apnea or central sleep apnea and a determination of whether the patient was non-complaint with CPAP during the sleeping event. For example, in response to a determination that a patient was non-complaint with CPAP during the sleeping event, a risk of suffering an arrhythmia increases. In some examples, in response to determination the sleep apnea burden was severe for a sleeping event, a risk of patient suffering AF increases the next day.

In some examples, processing circuitry 412 may be configured to determine at least one of a HF risk score, SCA risk score, arrhythmia risk score, or COPD risk score based, at least in part, on at least one of a sleep apnea burden of the previous night, a day-to-day trend in sleep apnea burden, use of CPAP, and whether the sleep apnea episodes were determined to be obstructive sleep apnea episodes of central sleep apnea episodes. In some examples, in response to determining whether patient was non-complaint with CPAP during the period of time, processing circuitry 412 may modify at least one of a HF risk score, SCA risk score, arrhythmia risk score, or COPD risk score based, at least in part, of a sleep apnea burden of the previous night. In some examples, processing circuitry 412 may be configured to determine whether patient 102 is in condition for AF ablation based, at least in part, on at least one of a sleep apnea burden of the previous night, a day-to-day trend in sleep apnea burden, use of CPAP, and whether the sleep apnea episodes were determined to be obstructive sleep apnea episodes of central sleep apnea episodes.

The techniques described herein, help enable long-term sleep apnea monitoring using an implantable medical device 406, such as an ICM. In some examples, the detected apnea episodes may be differentiated as obstructive or central sleep apnea using features determined from heart rate variation patterns including duration of the heart rate variation, magnitude of heart rate change and consecutiveness of the apnea episodes. Based on the detected apnea episodes, processing circuitry 412 may be configured to determine at least one of a nightly sleep apnea burden and night-to-night variabilities in the sleep apnea severity. In some examples, these determinations and these trends may be used to generate risk scores for COPD, HF, SCA events, AF, and occurrence of various arrhythmias. In some examples, the techniques may also be used to correlate adherence to CPAP with nightly sleep apnea burden severity. In some examples, personalized sleep apnea burden threshold may be provided for patients based on historic sleep apnea trends that may provide an improved personalized classification of severity of sleep apnea burden which may lead to improved accuracy in generation of risk scores for COPD, HF, SCA events, AF, and occurrence of various arrhythmias.

In some examples, some of the settings or thresholds described herein relating to the when and how processing circuitry 412 determine a classification a sleep apnea burden based on amount of detected sleep apnea episodes during a period of time may be programmable and tuned individually for each patient.

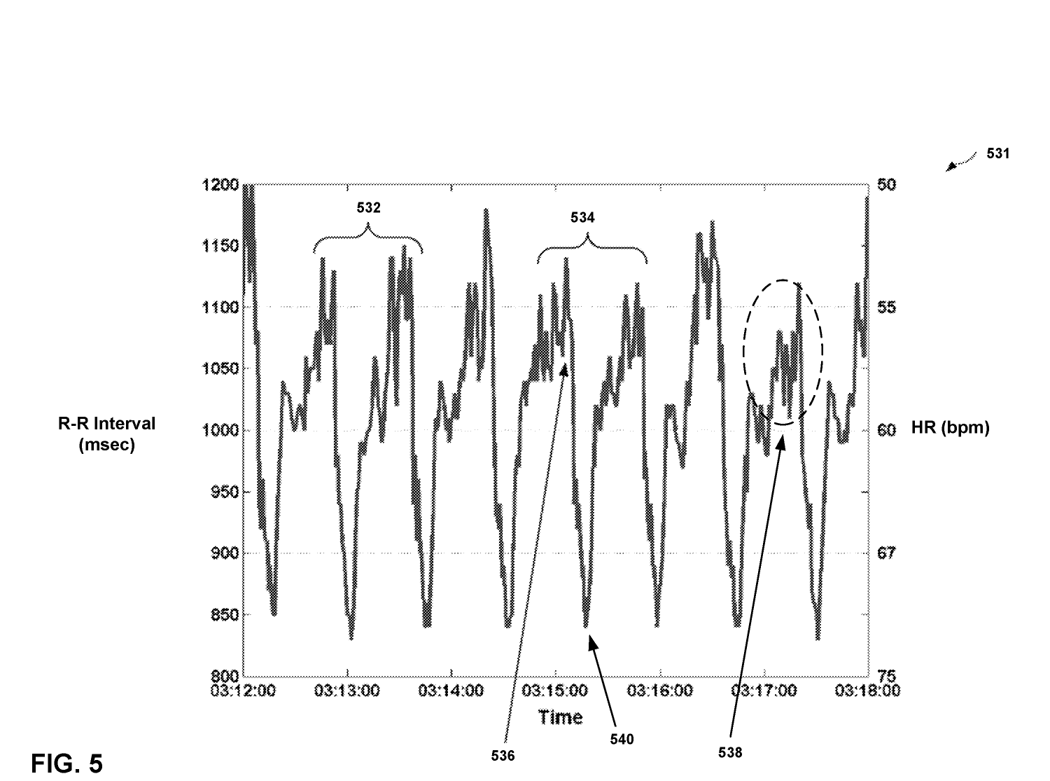

FIG. 5 is a graph 531 that illustrates sleep disordered heart rate (SDHR) information for patient. In FIG. 5, the R-R interval corresponds to the interval between two successive R-waves. In the example of graph 531, the SDHR of patient exhibits, at various times, characteristics of different physiological conditions. For instance, processing circuitry may analyze trends shown by clusters of maxima and minima (e.g., “peak” and “valley”) points of graph 531 to determine whether patient exhibits symptoms of a sleep apnea episode. For example, the processing circuitry of medical device 106 may use heart rate to the SDHR information in accordance with techniques of this disclosure. Some peak-and-valley clusters that the processing circuitry may analyze to detect possible sleep apnea episodes with respect to a patient are identified in FIG. 5.

For example, even during normal breathing, the heart rate information of graph 531 illustrates a type of cyclical variable heart rate (CVHR) referred to as respiratory sinus arrhythmia (RSA). During RSA, the heart rate of the patient increases with an inspiration (or inhalation) and the heart rate of patient decreases with expiration (or exhalation). RSA is associated with a single breath cycle, e.g., a single heart rate increase with inspiration and a single heart rate decrease with expiration. RSA is illustrated in the graph of FIG. 5 by higher frequency and lower amplitude variations 538.

The lower frequency and higher amplitude variations of the graph of FIG. 5, on the other hand, illustrate a form of CVHR, referred to herein as SDHR, associated with sleep apnea. For example, each of areas 532 and 534 illustrates one apnea arousal cycle, and the corresponding decrease and increase in heart rate. In most patients, a decrease of heart rate is observed during each apnea episode followed by an increase of heart rate near the end of the apnea episode. The heart rate further increases during ensuing hyperventilation. In the example of the individual sleep apnea episode associated with area 534 of graph 531, the heart rate increase due to autonomic arousal is indicated by crest 536. For instance, crest 536 may indicate a decrease that heart 104 experiences as the autonomic nervous system of patient attempts to compensate for the increased oxygen saturation during recovery from a sleep apnea episode. As such, troughs between such crests may be indicative of a period of overexertion of heart 104 which results from compensatory mechanisms that the autonomic nervous system of patient implements to counter the decreased oxygen saturation caused by the sleep apnea episode. As shown in FIG. 5, near the end of the sleep apnea episode (e.g., after a valley 540), the heart rate may increase.

Again, processing circuitry may collect and analyze physiological parameter value(s), determined in any way, to identify sleep apnea episodes based on the characteristics illustrated by the graph 531 of FIG. 5.

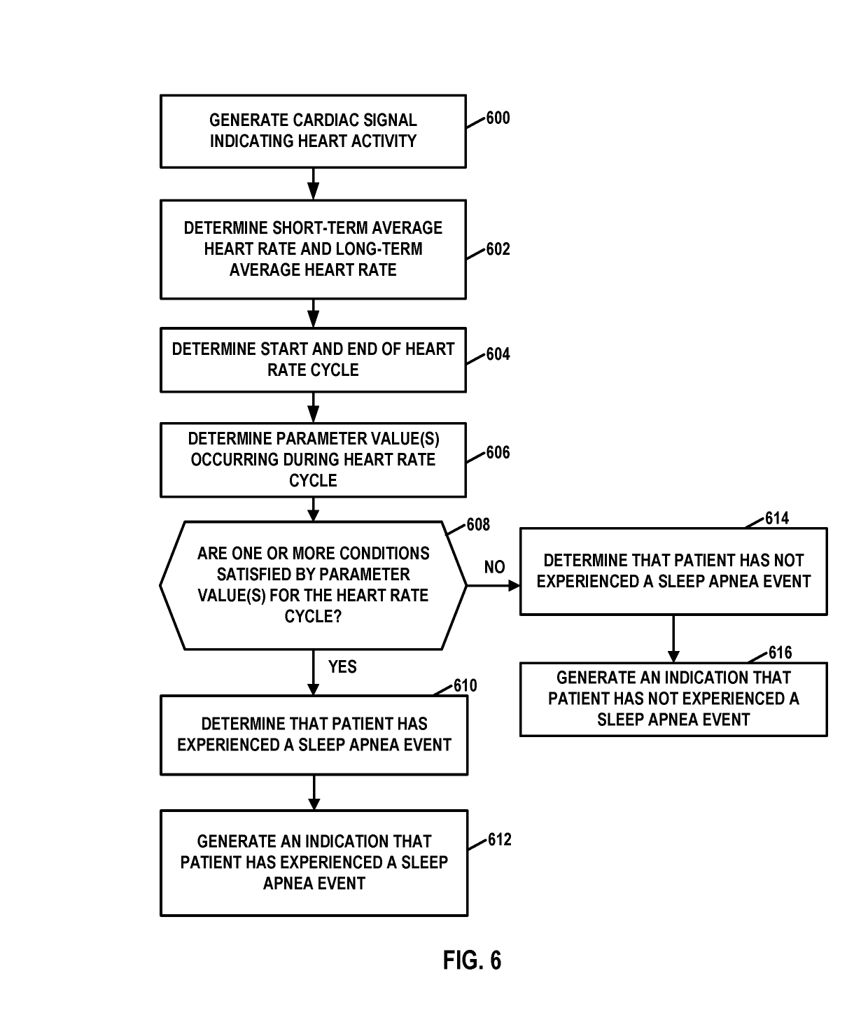

FIG. 6 is a flowchart illustrating a method by which a medical device system may use cardiac signal to detect a sleep apnea episode. Although described as being performed by medical device 406, one or more of the various example techniques described with reference to FIG. 6 may be performed by any one or more devices and/or systems including processing circuitry in accordance with techniques of this disclosure.

Medical device 406 may generate, by processing circuitry 412, a cardiac signal indicating activity of a heart of patient 102 in response to signals sensed by sensing circuitry 418 of medical device 406 (600). Medical device 406 may include one or more sensors 422, electrodes 430, etc., configured to sense signals produced by heart activity. Sensing circuitry 418 may deliver (e.g., send, transmit, etc.) detected signals to processing circuitry 412. Processing circuitry 412 may then generate a cardiac signal indicating the activity of a heart of patient 102. In some examples, the cardiac signal may be associated with characteristics of the heart activity of patient 102. For example, the cardiac signal may be associated with the waves, intervals, durations, and rhythm of the heart activity of patient 102.

Processing circuitry 412 may determine a short-term average heart rate and a long-term average heart rate upon which the start and end of a heart rate cycle may be based (602). The short-term average of the heart rate of patient 102 may be based on a first number of heartbeats, and the long-term average of the heart rate of patient 102 may be based on a second number of heartbeats, wherein the first number is less than the second number. For example, the first number of heartbeats may be equal to 3 heartbeats and the second number of heartbeats may be equal to 120 heartbeats. In such an example, the short-term average of the heart rate of patient 102 may be based on 3 heartbeats, and the long-term average of the heart rate of patient 102 may be based on 120 heartbeats. In other examples, the first number of heartbeats may be equal to a value other than 3 (e.g., 4, 5, etc.) and/or the second number of heartbeats may be equal to a value other than 120 (e.g., 119, 130, etc.).

Processing circuitry 412 may calculate the short-term average of the heart rate and the long-term average of the heart rate in various ways. For example, the short-term average of the heart rate and the long-term average of the heart rate may be the median of the first number of heartbeats and the second number of heartbeats, respectively (e.g., the median of 3 heartbeats and the median of 120 heartbeats, respectively). Alternatively, the short-term average of the heart rate and the long-term average of the heart rate may be the mean of the first number of heartbeats and the second number of heartbeats, respectively (e.g., the mean of 3 heartbeats and the mean of 120 heartbeats, respectively). Alternatively, the short-term average of the heart rate and the long-term average of the heart rate may be the mode of the first number of heartbeats and the second number of heartbeats (e.g., the mode of 3 heartbeats and the mode of 120 heartbeats, respectively). It should be understood that other methods for calculating the short-term average of the heart rate and the long-term average of the heart rate may be appropriate depending upon the circumstances.

Processing circuitry 412 may further determine the start and end of a heart rate cycle (604). The heart rate cycle defines a period of time of the patient 102’s heart activity that is being processed by processing circuitry 412 to detect the occurrence of a sleep apnea episode. The start of the heart rate cycle may be based on a first time the short-term average of the heart rate of patient 102 changes from being less than the long-term average of the heart rate of patient 102 to being greater than the long-term average of the heart rate of patient 102. For example, if the long-term average of the heart rate of patient 102 is a constant 65 beats per minute (BPM), then the start of the heart rate cycle may be determined based on the first time that the short-term average of the heart rate of patient 102 exceeds 65 BPM. Similarly, the end of the heart rate cycle may be based on the second time the short-term average of the heart rate of patient 102 changes from being less than long-term average of the heart rate of patient 102 to being greater than the long-term average of the heart rate of patient 102. For example, if the long-term average of the heart rate of patient 102 is a constant 65 BPM, then the end of the heart rate cycle may be determined based on the second time that the short-term average of the heart rate of patient 102 exceeds 65 BPM.

Processing circuitry 412 may further determine corresponding parameter values for one or more heart rate parameters of patient 102 based on heart rate (and associated activity) occurring during the heart rate cycle (606). Determining whether patient 102 has experienced a sleep apnea episode during the first period may be based on the parameter values for the one or more heart rate parameters.

The parameter values determined by processing circuitry 412 may include a peak-to-valley time interval. The peak-to-valley time interval may be the time interval between a maximum short-term average of the heart rate during the heart rate cycle and a minimum short-term average of the heart rate during the heart rate cycle. For example, if a maximum short-term average of the heart rate value of 75 BPM occurs 30 seconds from a reference point (e.g., the beginning of a recording of the heart rate cycle, the beginning of the heart rate cycle, etc.) and during the heart rate cycle and the minimum short-term heart rate average value of 60 BPM occurs 45 seconds from the reference point and during the heart rate cycle, then the peak-to-valley time interval is equal to 15 seconds.

The parameter values determined by processing circuitry 412 may further include an activity count. The activity count for the heart rate cycle may indicate a number of time intervals during the heart rate cycle in which an amount of movement of the patient 102 is greater than a minimum movement threshold. Amount of movement of patient 102 may be determined using one or more sensors 422 (e.g., accelerometer 424). For example, accelerometer 424 may measure the acceleration of patient’s body, and if the acceleration of patient’s body exceeds an acceleration threshold corresponding to a minimum movement threshold, the activity count for the heart rate cycle may be incremented by one. As such, if, in a single heart rate cycle, the acceleration of patient’s body exceeds the acceleration threshold 8 times, the minimum movement threshold may also be exceeded 8 times so that the activity count for patient 102 is 8.

The parameter values determined by processing circuitry 412 may further include a peak-to-valley heart rate variation value. The peak-to-valley heart rate variation value for the heart rate cycle may be the difference between a maximum short-term average of the heart rate during the heart rate cycle and a minimum short-term average of the heart rate during the heart rate cycle. For example, if the maximum short-term average of the heart rate during the heart rate cycle is 75 BPM and the minimum short-term average of the heart rate during the heart rate cycle is 60 BPM, then the peak-to-valley heart rate variation value is 15 BPM for this heart rate cycle.

The parameter values determined by processing circuitry 412 may further include a cycle length, where the cycle length for the heart rate cycle indicates a length of the heart rate cycle. For example, if the start of the heart rate cycle is at first time of 0 seconds (e.g., based on the first time that the short-term average of the heart rate of patient 102 exceeds the long-term average of the heart rate of patient 102), and the end of the heart rate cycle is at second time of 70 seconds (e.g., based on the second time that the short-term average of the heart rate of patient 102 exceeds the long-term average of the heart rate of patient 102), the cycle length is 70 seconds.

In some examples, processing circuitry 412 may derive a parameter value from other parameter values. For example, processing circuitry 412 may use a series of heart rate values to determine the peak-to-valley time interval, which in turn may be used to detect a sleep apnea episode. For example, if the short-term average of the heart rate of patient 102 fluctuates from 70 BPM to 60 BPM, 60 BPM to 85 BPM, 85 BPM to 65 BPM, and so on during the heart cycle, heart rate values may include 10 BPM (70 BPM less 60 BPM), 25 BPM (85 BPM less 60 BPM), 5 BPM (70 BPM less 65 BPM), and so on. In this example, the peak-to-valley variation value is 25 BPM because 85 BPM is the maximum short-term heart rate average value during the heart rate cycle, and 60 BPM is the minimum short-term average value during the heart rate cycle.

Processing circuitry 412 may then use the respective times that the maximum short-term average of the heart rate value of 85 BPM and the minimum short-term average of the heart rate value of 60 BPM occurred to determine the peak-to-valley time interval. In some examples, the peak-to-valley time interval may equal the time the valley occurred subtracted by the time the peak occurred. For example, if the maximum short-term average of the heart rate value of 85 BPM occurred 30 seconds from the start of the heart rate cycle and the minimum short-term average of the heart rate value of 60 BPM occurred 60 seconds from the start of the heart rate cycle, then the peak-to-valley time interval is equal to 30 seconds.

Processing circuitry 412 may further determine whether one or more conditions of a plurality of conditions are satisfied by one or more parameter values for patient 102 for the heart rate cycle. The plurality of conditions may include, but is not limited to, a peak-to-valley time interval condition, an activity count condition, a peak-to-valley heart rate variation condition, a cycle length condition, etc.

The plurality of conditions may include a peak-to-valley time interval condition. The peak-to-valley time interval condition may be a condition that the peak-to-valley time interval is greater than a lower peak-to-valley time threshold and less than an upper peak-to-valley time threshold. For example, if the lower peak-to-valley time threshold is equal to 5 seconds and the upper peak-to-valley time threshold is equal to 30 seconds, then processing circuitry 412 may determine that the peak-to-valley time interval condition is satisfied if the peak-to-valley time interval is greater than 5 seconds and less than 30 seconds. As such, a peak-to-valley time interval of 10 seconds, for example, would satisfy the peak-to-valley time interval condition. Alternatively, processing circuitry 412 may determine that the peak-to-valley time interval condition is not satisfied if the peak-to-valley time interval is not greater than 5 seconds and less than 30 seconds. As such, a peak-to-valley time interval of 3 seconds, for example, would not satisfy the peak-to-valley time interval condition.

Additionally or alternatively, the plurality of conditions may include an activity count condition. The activity count condition may be a condition that an activity count for the heart rate cycle is less than an activity count threshold. For example, if the activity count threshold is equal to 8, then processing circuitry 412 may determine that the activity count condition is satisfied if the activity count is less than 8. As such, an activity count of 5, for example, would satisfy the activity count condition. Alternatively, processing circuitry 412 may determine that the activity count condition is not satisfied if the activity count is not less than 8. As such, an activity count of 10, for example, would not satisfy the activity count condition.

Additionally or alternatively, the plurality of conditions may include a peak-to-valley HRV condition. The peak-to-valley HRV condition may be a condition that the peak-to-valley HRV value for the heart rate cycle is greater than a lower peak-to-valley heart rate variation threshold and less than an upper peak-to-valley heart rate variation threshold. For example, if the lower peak-to-valley heart rate variation threshold is equal to 6 BPM and the upper peak-to-valley heart rate variation threshold is equal to 50 BPM, then processing circuitry 412 may determine that the peak-to-valley HRV condition is satisfied if the peak-to-valley HRV value is greater than 6 and less than 50. As such, a peak-to-valley HRV value of 25, for example, would satisfy the peak-to-valley HRV condition. Alternatively, processing circuitry 412 may determine that the peak-to-valley HRV condition is not satisfied if the peak-to-valley HRV value is not less than 50. As such, a peak-to-valley HRV value of 60, for example, would not satisfy the peak-to-valley HRV condition.

Additionally or alternatively, the plurality of conditions may include a cycle length condition. The cycle length is the length of the heart rate cycle (e.g., the length of the period of time of patient 102’s heart activity being processed by processing circuitry 412 for detecting a sleep apnea episode). The cycle length condition may be a condition that the cycle length for the heart rate cycle is greater than a lower cycle length threshold and less than an upper cycle length threshold. For example, if the lower cycle length threshold is equal to 25 seconds and the upper cycle length threshold is equal to 100 seconds, then processing circuitry 412 may determine that the cycle length condition is satisfied if the cycle length is greater than 25 seconds and less than 100 seconds. As such, a cycle length of 50 seconds, for example, would satisfy the cycle length condition. Alternatively, processing circuitry 412 determine that the cycle length condition is not satisfied if the cycle length is not greater than 25 seconds and less than 100 seconds. As such, a cycle length of 125 seconds, for example, would not satisfy the cycle length condition.

In some examples, the plurality of conditions may include the peak-to-valley time interval condition, the activity count condition, the peak-to-valley heart rate variation condition, and the cycle length condition. In such examples, determining that the patient has experienced the sleep apnea episode may be based at least in part on each of the conditions of the plurality of conditions being satisfied for the heart rate cycle.

In some examples, processing circuitry 412 may further determine whether a patient 102 has experienced a sleep apnea episode based on whether one or more conditions of the plurality of conditions are satisfied (608). For example, if each of the one or more conditions is satisfied (e.g., peak-to-valley time interval condition is satisfied) (“YES” branch of 608), processing circuitry 412 may determine that patient 102 has experienced a sleep apnea episode or that the probability that patient 102 has experienced a sleep apnea episode is not low (610). Processing circuitry 412 may then cause the generation of an indication that patient 102 has experienced a sleep apnea episode (612). For example, processing circuitry 412 may cause medical device 406 and/or external device 408 to generate an indication that patient 102 has experienced a sleep apnea episode.

Alternatively, if one or more of the conditions have not been satisfied (“NO” branch of 908), processing circuitry 412 may determine that patient 102 has not experienced a sleep apnea episode or that the probability that patient 102 has experienced a sleep apnea episode is low (614). Processing circuitry 412 may then cause the generation of an indication that patient 102 has not experienced a sleep apnea episode (616). For example, processing circuitry 412 may cause medical device 406 and/or external device 408 to generate an indication that patient 102 has not experienced a sleep apnea episode. Alternatively, in some examples, processing circuitry 412 does not generate an indication that patient 102 has experienced a sleep apnea episode if patient 102 has not experienced a sleep apnea episode.

Processing circuitry 412 may generate an indication that patient 102 has experienced a sleep apnea episode. For example, processing circuitry 412 may cause the medical device or an external device to output an indication that patient 102 has experienced a sleep apnea episode. The external device may include one or more cellular phones, a ‘smartphone,’ a satellite phone, a notebook computer, a tablet computer, a wearable device, a computer workstation, a personal digital assistant, a handheld computing device, a virtual reality headset, or any other device that may output an indication that patient 102 has experienced a sleep apnea episode. External device may output the indication automatically (e.g., at a pre-determined time of day) and/or in response to input from patient 102 as part of a report or history of patient and in the form of an audible notification, a visual notification, a tactile notification (e.g., a vibration or vibrational pattern), a text prompt, a button prompt, and/or any other notification that may indicate to patient 102 that patient 102 has experienced a sleep apnea episode.

Although not illustrated in FIG. 6, processing circuitry 412 may determine that patient 102 has experienced a sleep apnea episode based on one or more conditions being satisfied by at least a non-zero minimum number of additional heart rate cycles other than the current heart rate cycle that occur within a predetermined temporal distance (e.g., amount of time) of the current heart rate cycle. For example, if the minimum number of additional heart rate cycles is equal to 2 and the predetermined temporal distance is equal to 240 seconds, then processing circuitry 412 may determine that patient 102 has experienced a sleep apnea episode based on one or more conditions (e.g., the peak-to-valley time interval condition, the activity count condition, the peak-to-valley heart rate variation condition, the cycle length condition, etc.) being satisfied by 2 additional heart rate cycles other than the current heart rate cycle that occur within 240 seconds of the current heart rate cycle. As such, processing circuitry 412 may determine that patient 102 has experienced a sleep apnea episode based on the activity count condition being satisfied by a heart rate cycle with a temporal distance of 220 seconds of the current heart rate cycle and the peak-to-valley heart rate variation condition being satisfied by a heart rate cycle with a temporal distance of 75 seconds of the current heart rate cycle. Alternatively, processing circuitry 412 may determine that patient 102 has not experienced a sleep apnea episode based on the activity count condition being satisfied by a heart rate cycle with a temporal distance of 300 seconds of the current heart rate cycle and the peak-to-valley heart rate variation condition being satisfied by a heart rate cycle with a temporal distance of 75 seconds of the current heart rate cycle because the heart rate cycle with a temporal distance of 300 seconds did not occur within the predetermined temporal distance of 240 seconds. Additionally or alternatively, in the same example, processing circuitry 412 may determine that patient 102 has not experienced a sleep apnea episode because one or more conditions were not satisfied by 2 or more additional heart rate cycles (e.g., one condition was only satisfied by 1 additional heart rate cycle because only the activity count condition was satisfied by only 1 heart rate cycle with a temporal distance of 75 seconds of the current heart rate cycle).

The reference point for determining the temporal distance may vary. For example, the reference point for determining the temporal distance may be the start of a heart rate cycle, the end of the heart rate cycle, and any point in between the start and end of the heart rate cycle. In some examples, the direction of the temporal distance may vary. That is, the direction of the temporal distance may be retrospective so that heart activity that already occurred may be evaluated, prospective so that heart activity that will occur may be evaluated, or a combination of both.

For example, if the reference point for determining the temporal distance is the start of the heart rate cycle and the predetermined temporal distance is 240 seconds, then processing circuitry 412 may determine that patient 102 has experienced a sleep apnea episode based on heart activity that occurred within any heart rate cycle before the start of the current heart rate cycle with a temporal distance of 240 seconds or less. In another example, processing circuitry 412 may determine that patient 102 has experienced a sleep apnea episode based on heart activity that occurred within any heart rate cycle after the start of the current heart rate cycle with a temporal distance of 240 seconds or less. In yet another example, processing circuitry 412 may determine that patient 102 has experienced a sleep apnea episode based on heart activity occurring within any heart rate cycle before the start of the current heart rate cycle and after the start of the current heart cycle, as long as the heart activity satisfying the one or more conditions occur within a temporal distance of 240 seconds or less.

In another example, if the reference point for determining the temporal distance is the end of the heart rate cycle and the predetermined temporal distance is 240 seconds, then processing circuitry 412 may determine that patient 102 has experienced a sleep apnea episode based on heart activity that occurred within any heart rate cycle before the end of the current heart rate cycle with a temporal distance of 240 seconds or less. Alternatively, processing circuitry 412 may determine that patient 102 has experienced a sleep apnea episode based on heart activity that occurred within any heart rate cycle after the end of the current heart rate cycle with a temporal distance of 240 seconds or less. Alternatively, processing circuitry 412 may determine that patient 102 has experienced a sleep apnea episode based on heart activity that occurred within any heart rate cycle before the end of the current heart rate cycle with a temporal distance of 120 seconds or less and within any heart rate cycle after the end of the current heart rate cycle with a temporal distance of 120 seconds or less.

In another example, if the reference point for determining the temporal distance is the midpoint of the heart rate cycle and the predetermined temporal distance is 240 seconds, then processing circuitry 412 may determine that patient 102 has experienced a sleep apnea episode based on heart activity that occurred within any heart rate cycle before the midpoint of the current heart rate cycle with a temporal distance of 240 seconds or less. Alternatively, processing circuitry 412 may determine that patient 102 has experienced a sleep apnea episode based on heart activity that occurred within any heart rate cycle after the midpoint of the current heart rate cycle with a temporal distance of 240 seconds or less. Alternatively, processing circuitry 412 may determine that patient 102 has experienced a sleep apnea episode based on heart activity that occurred within any heart rate cycle before the midpoint of the current heart rate cycle with a temporal distance of 120 seconds or less and within any heart rate cycle after the midpoint of the current heart rate cycle with a temporal distance of 120 seconds or less.

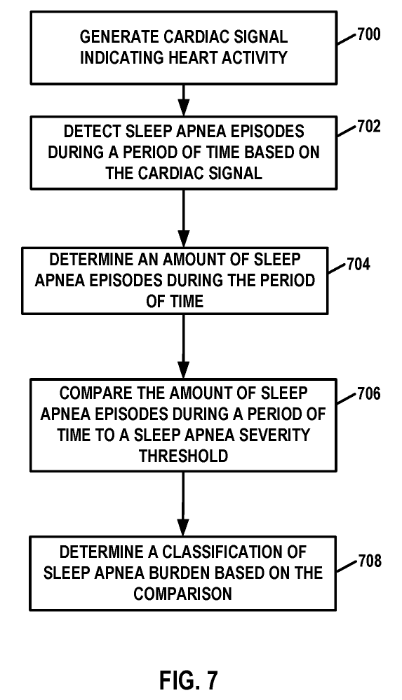

FIG. 7 is a flowchart illustrating a method by which a medical device system monitor patient health based on sleep apnea episode detection. Although described as being performed by medical device 406, one or more of the various example techniques described with reference to FIG. 6 may be performed by any one or more devices and/or systems including processing circuitry in accordance with techniques of this disclosure.

Medical device system 406 may generate, by processing circuitry 412, a cardiac signal indicating activity of a heart of patient 102 in response to signals sensed by sensing circuitry 418 of medical device 406 (700). Processing circuitry 412 may detect one or more sleep apnea episodes during a period of time based on the cardiac signal (702). In some examples, as described above (e.g., with respect to FIG. 6), medical device 406 may use heart rate to determine sleep apnea episodes during a period of time.

Processing circuitry 412 may determine an amount of sleep apnea episodes during the period of time (704). In some examples, processing circuitry 412 may determine an amount of sleep apnea episodes occurring in each of a plurality of periods of time. In some examples, processing circuitry 412 may determine an amount of sleep apnea episodes occurring in each of a plurality of periods of time during a nighttime sleeping event (e.g., an amount of sleep apnea episodes occurring during over each period of time, such as 1-hour periods of time).

Processing circuitry 412 may compare the amount of sleep apnea episodes during a period of time to a sleep apnea severity threshold (706). In some examples, a sleep apnea severity threshold may include a plurality of threshold levels, such as “mild”, “moderate”, and “severe.” In some examples, a severe sleep apnea burden threshold may be at least 30 detected sleep apnea episodes during the period of time. In some examples, a severe sleep apnea burden threshold may be at least 35 detected sleep apnea episodes during the period of time. In some examples, a mild sleep apnea burden threshold may be 15 or less detected sleep apnea episodes during the period of time. For example, processing circuitry may determine the classification of the detected sleep apnea episodes is severe based on the amount of detected sleep apnea episodes satisfying the severe sleep apnea burden threshold (e.g., at least 30 sleep apnea episodes during the period of time) or may determine the classification of the detected sleep apnea episodes is mild based on the amount of detected sleep apnea episodes satisfying the mild sleep apnea burden threshold (e.g., detecting 15 or less sleep apnea episodes during the period of time).

Processing circuitry 412 may determine a classification of sleep apnea burden for the patient based on the comparison (708). In some examples, processing circuitry 412 may determine the classification of sleep apnea burden is severe based on the amount of detected sleep apnea episodes being greater than a severe sleep apnea burden threshold (e.g., the amount of detected sleep apnea episodes during the period of time corresponding to being greater than or equal to 30 sleep apnea episodes per hour). In some examples, processing circuitry 412 may determine a change in daily sleep apnea burden over a period of time, such as over a plurality of days. In some examples, processing circuitry 412 may be configured to determine at least one of a HF risk score, SCA risk score, arrhythmia risk score, or COPD risk score based, at least in part, on at least one of a sleep apnea burden of the previous night or a day-to-day trend in sleep apnea burden.

In some examples, processing circuitry 412 may be configured to classify at least one sleep apnea episode of the one or more sleep apnea episodes as obstructive sleep apnea or central sleep apnea based on at least one of the magnitude of heart rate change during the sleep apnea episode or the duration of heart rate change during the sleep apnea episodes. In some examples, processing circuitry 412 may be able to determine whether patient had compliant use with CPAP during a nighttime sleep event, such as by receiving an indication from a user input into a patient computing device.

In some examples, processing circuitry 412 may be configured to determine at least one of a HF risk score, SCA risk score, arrhythmia risk score, or COPD risk score based, at least in part, on at least one of a sleep apnea burden of the previous night or a day-to-day trend in sleep apnea burden, patient use of CPAP, and whether the sleep apnea episodes were determined to be obstructive sleep apnea episodes of central sleep apnea episodes

In some examples, 412 may detect sleep apnea episodes based on heart rate by implementing a machine learned model or other machine learned or artificial intelligence (AI) algorithm. Example machine learning techniques that may be employed to generate such algorithms can include various learning styles, such as supervised learning, unsupervised learning, and semi-supervised learning. Example types of algorithms include Bayesian algorithms, Clustering algorithms, decision-tree algorithms, regularization algorithms, regression algorithms, instance-based algorithms, artificial neural network algorithms, deep learning algorithms, dimensionality reduction algorithms and the like. Various examples of specific algorithms include Bayesian Linear Regression, Boosted Decision Tree Regression, and Neural Network Regression, Back Propagation Neural Networks, Convolution Neural Networks (CNN), Long Short Term Networks (LSTM), the Apriori algorithm, K-Means Clustering, k-Nearest Neighbour (kNN), Learning Vector Quantization (LVQ), Self-Organizing Map (SOM), Locally Weighted Learning (LWL), Ridge Regression, Least Absolute Shrinkage and Selection Operator (LASSO), Elastic Net, and Least-Angle Regression (LARS), Principal Component Analysis (PCA) and Principal Component Regression (PCR).

The input(s) to such an algorithm may include one or more vectors of data, such as EGM or ECG signal samples, values of the heart rate and/or HRV parameters described herein, or impedance signal samples. An output of such an algorithm may be a value representing a likelihood, probability, and/or confidence of one or more classifications, such as “sleep apnea” or “not sleep apnea.” As one example, such an algorithm may include a CNN or other deep learning model trained with numerous training data samples labeled as “sleep apnea” or “not sleep apnea.”

As used herein, an implantable medical device (IMD) may include, be, or be part of a variety of devices or integrated systems, such as, but not limited to, implantable cardiac monitors (ICMs), implantable pacemakers, including those that deliver cardiac resynchronization therapy (CRT), implantable cardioverter defibrillators (ICDs), diagnostics device, cardiac device, etc. Various examples have been described that include detecting episodes of sleep apnea using cardiac cycle length metrics. In addition, pulmonary therapy may be provided to mitigate the severity of the sleep apnea episode or counter the effects of the sleep apnea episode. Any combination of detection and therapy for sleep apnea episodes is contemplated.

Various aspects of the techniques may be implemented within one or more processing circuitries, including one or more microprocessors, DSPs, ASICs, FPGAs, or any other equivalent integrated or discrete logic circuitry, as well as any combinations of such components, embodied in external devices, such as clinician or patient external devices, electrical stimulators, or other devices. The term “processing circuitry” may generally refer to any of the foregoing logic circuitry, alone or in combination with other logic circuitry or any other equivalent circuitry.

In one or more examples, the functions described in this disclosure may be implemented in hardware, software, firmware, or any combination thereof. If implemented in software, the functions may be stored on, as one or more instructions or code, a computer-readable medium and executed by a hardware-based processing unit. Computer-readable media may include computer-readable storage media forming a tangible, non-transitory medium. Instructions may be executed by one or more processing circuitries, such as one or more DSPs, ASICs, FPGAs, general purpose microprocessors, or other equivalent integrated or discrete logic circuitry. Accordingly, the term “processing circuitry,” as used herein may refer to one or more of any of the foregoing structure or any other structure suitable for implementation of the techniques described herein.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, including an IMD, an external device, a combination of an IMD and external device, an integrated circuit (IC) or a set of ICs, and/or discrete electrical circuitry, residing in an IMD and/or external device.

The following examples are illustrative of the techniques described herein.

Example 1: A medical device includes sensing circuitry configured to sense a cardiac signal indicating activity of a heart of a patient; and processing circuitry configured to: detect one or more sleep apnea episodes of the patient occurring during a period of time based on the cardiac signal; compare an amount of detected sleep apnea episodes of the patient during the period of time to a sleep apnea severity threshold; and determine a classification of sleep apnea burden of the patient based on the comparison.

Example 2: The medical device of example 1, wherein the sleep apnea burden is with respect to a nighttime sleeping event including the period of time.

Example 3: The medical device of example 1, wherein the period of time is for at least 1 hour.

Example 4: The medical device of any of examples 1-3, wherein the processing circuitry is further configured to: determine the classification of sleep apnea burden is severe sleep apnea burden based on the amount of detected sleep apnea episodes being greater than a severe sleep apnea burden threshold.

Example 5: The medical device of example 4, wherein the severe sleep apnea burden threshold is at least 30 sleep apnea episodes during the period of time.

Example 6: The medical device of example 4, wherein the processing circuitry is further configured to: determine the severe sleep apnea burden threshold based on historic sleep apnea trends of the patient.

Example 7: The medical device of example 6, wherein the processing circuitry is further configured to: store at least some of the historic sleep apnea trends of the patient in a memory.

Example 8: The medical device of any of examples 1-7, wherein the processing circuitry is further configured to: determine at least one of a heart failure (HF) risk score, sudden cardiac arrest (SCA) risk score, arrhythmia risk score, or chronic obstructive pulmonary disease (COPD) risk score based, at least in part, on the classification of the sleep apnea burden.

Example 9: The medical device of any of examples 1-8, wherein the processing circuitry is further configured to: determine at least one of a magnitude of heart rate change or a duration of heart rate change based on the cardiac signal; and classify at least one of the detected sleep apnea episodes as obstructive sleep apnea or central sleep apnea based on at least one of the magnitude of heart rate change or the duration of heart rate change.

Example 10: The medical device of example 9, wherein the processing circuitry is further configured to: determine at least one of a heart failure (HF) risk score, sudden cardiac arrest (SCA) risk score, arrhythmia risk score, or chronic obstructive pulmonary disease (COPD) risk score based, at least in part, on the classification of the sleep apnea burden and the classification of the at least one of the detected sleep apnea episodes as obstructive sleep apnea or central sleep apnea.

Example 11: The medical device of any of examples 1-7, wherein the processing circuitry is further configured to: determine whether patient was non-complaint with continuous positive airway pressure (CPAP) during the period of time; and determine a classification of sleep apnea burden during the period of time based on the comparison and the determination of whether the patient was non-complaint with CPAP during the period of time.

Example 12: The medical device of any of examples 1-7, wherein the processing circuitry is further configured to: determine whether patient was non-complaint with continuous positive airway pressure (CPAP) during the period of time; and determine at least one of a heart failure (HF) risk score, sudden cardiac arrest (SCA) risk score, arrhythmia risk score, or chronic obstructive pulmonary disease (COPD) risk score based, at least in part, on the classification of the sleep apnea burden and whether the patient was non-complaint with CPAP during the period of time.

Example 13: The medical device of any of examples 1-7, wherein the processing circuitry is further configured to: determine a plurality of classifications of sleep apnea burden over a respective plurality of days; and determine a daily change in sleep apnea burden for the patient based on the determined plurality of classifications of sleep apnea burden over the respective plurality of days.

Example 14: The medical device of example 13, wherein the processing circuitry is further configured to determine at least one of a heart failure (HF) risk score, sudden cardiac arrest (SCA) risk score, arrhythmia risk score, or chronic obstructive pulmonary disease (COPD) risk score based, at least in part, on the daily change in sleep apnea burden.

Example 15: The medical device of any of examples 1-14, wherein the processing circuitry is further configured to: determine heart rates of the patient based on the cardiac signal sensed during the period of time; and detect the one or more sleep apnea episodes of the patient occurring during the period of time based on the heart rates.

Example 16: A method includes sensing, by sensing circuitry of a medical device, a cardiac signal indicating activity of a heart of a patient; detecting, by processing circuitry, one or more sleep apnea episodes of the patient occurring during a period of time based on the cardiac signal; comparing, by the processing circuitry, an amount of detected sleep apnea episodes of the patient during the period of time to a sleep apnea severity threshold; and determining, by the processing circuitry, a classification of sleep apnea burden during the period of time based on the comparison.

Example 17: The method of example 16, wherein the period of time is a nighttime sleeping event including the period of time.

Example 18: The method of example 16, wherein the period of time is for at least 1 hour.

Example 19: The method of any of examples 16-18, further includes determining, by the processing circuitry, the classification of sleep apnea burden is severe sleep apnea burden based on the amount of detected sleep apnea episodes being greater than a severe sleep apnea burden threshold.

Example 20: The method of example 19, wherein the severe sleep apnea burden threshold is at least 30 sleep apnea episodes during the period of time.

Example 21: The method of example 19, further includes determining, by the processing circuitry, the severe sleep apnea burden threshold based on historic sleep apnea trends of the patient.

Example 22: The method of example 21, further includes storing, by the processing circuitry, at least some of the historic sleep apnea trends of the patient in a memory.

Example 23: The method of any of examples 16-22, further includes determining, by the processing circuitry, at least one of a heart failure (HF) risk score, sudden cardiac arrest (SCA) risk score, arrhythmia risk score, or chronic obstructive pulmonary disease (COPD) risk score based, at least in part, on the classification of the sleep apnea burden.

Example 24: The method of any of examples 16-23, further includes determining, by the processing circuitry, at least one of a magnitude of heart rate change or a duration of heart rate change based on the cardiac signal; and classifying, by the processing circuitry, at least one of the detected sleep apnea episodes as obstructive sleep apnea or central sleep apnea based on at least one of the magnitude of heart rate change or the duration of heart rate change.

Example 25: The method of example 24, further includes determining, by the processing circuitry, at least one of a heart failure (HF) risk score, sudden cardiac arrest (SCA) risk score, arrhythmia risk score, or chronic obstructive pulmonary disease (COPD) risk score based, at least in part, on the classification of the sleep apnea burden and the classification of the at least one of the detected sleep apnea episodes as obstructive sleep apnea or central sleep apnea.

Example 26: The method of any of examples 16-23, further includes determining, by the processing circuitry, whether patient was non-complaint with continuous positive airway pressure (CPAP) during the period of time; and determining, by the processing circuitry, a classification of sleep apnea burden during the period of time based on the comparison and the determination of whether the patient was non-complaint with CPAP during the period of time.

Example 27: The method of any of examples 16-23, further includes determining, by the processing circuitry, whether patient was non-complaint with continuous positive airway pressure (CPAP) during the period of time; and determining, by the processing circuitry, at least one of a heart failure (HF) risk score, sudden cardiac arrest (SCA) risk score, arrhythmia risk score, or chronic obstructive pulmonary disease (COPD) risk score based, at least in part, on the classification of the sleep apnea burden and whether the patient was non-complaint with CPAP during the period of time.

Example 28: The method of any of examples 16-23, further includes determining, by the processing circuitry, a plurality of classifications of sleep apnea burden over a respective plurality of days; and determining, by the processing circuitry, a daily change in sleep apnea burden for the patient based on the determined plurality of classifications of sleep apnea burden over the respective plurality of days.

Example 29: The method of examples 28, further includes determining, by the processing circuitry, at least one of a heart failure (HF) risk score, sudden cardiac arrest (SCA) risk score, arrhythmia risk score, or chronic obstructive pulmonary disease (COPD) risk score based, at least in part, on the daily change in sleep apnea burden.

Example 30: The method of any of examples 16-23, further includes determining, by the processing circuitry of the medical device, heart rates of the patient based on the cardiac signal sensed during the period of time; and detecting, by the processing circuitry, the one or more sleep apnea episodes of the patient occurring during the period of time based on the heart rates.

Example 31: A computer-readable medium comprising instructions that, when executed, causes processing circuitry of a medical system to perform the method of any of examples 16-30.

Various examples have been described. These and other examples are within the scope of the following claims.

What is claimed is:

1. A medical device comprising:
sensing circuitry configured to sense a cardiac signal indicating activity of a heart of a patient; and
processing circuitry configured to:
detect one or more sleep apnea episodes of the patient occurring during a period of time based on the cardiac signal;
compare an amount of detected sleep apnea episodes of the patient during the period of time to a sleep apnea severity threshold; and
determine a classification of sleep apnea burden of the patient based on the comparison.

2. The medical device of claim 1, wherein the sleep apnea burden is with respect to a nighttime sleeping event including the period of time.

3. The medical device of claim 1, wherein the period of time is for at least 1 hour.

4. The medical device of claim 1, wherein the processing circuitry is further configured to:
determine the classification of sleep apnea burden is severe sleep apnea burden based on the amount of detected sleep apnea episodes being greater than a severe sleep apnea burden threshold.

5. The medical device of claim 4, wherein the severe sleep apnea burden threshold is at least 30 sleep apnea episodes during the period of time.

6. The medical device of claim 4, wherein the processing circuitry is further configured to:
determine the severe sleep apnea burden threshold based on historic sleep apnea trends of the patient.

7. The medical device of claim 6, wherein the processing circuitry is further configured to:
store at least some of the historic sleep apnea trends of the patient in a memory.

8. The medical device of claim 1, wherein the processing circuitry is further configured to:
determine at least one of a magnitude of heart rate change or a duration of heart rate change based on the cardiac signal; and
classify at least one of the detected sleep apnea episodes as obstructive sleep apnea or central sleep apnea based on at least one of the magnitude of heart rate change or the duration of heart rate change.

9. The medical device of claim 8, wherein the processing circuitry is further configured to:
determine at least one of a heart failure (HF) risk score, sudden cardiac arrest (SCA) risk score, arrhythmia risk score, or chronic obstructive pulmonary disease (COPD) risk score based, at least in part, on the classification of the sleep apnea burden and the classification of the at least one of the detected sleep apnea episodes as obstructive sleep apnea or central sleep apnea.

10. The medical device of claim 1, wherein the processing circuitry is further configured to:
determine whether patient was non-complaint with continuous positive airway pressure (CPAP) during the period of time; and
determine at least one of a heart failure (HF) risk score, sudden cardiac arrest (SCA) risk score, arrhythmia risk score, or chronic obstructive pulmonary disease (COPD) risk score based, at least in part, on the classification of the sleep apnea burden and whether the patient was non-complaint with CPAP during the period of time.

11. The medical device of claim 1, wherein the processing circuitry is further configured to:
determine a plurality of classifications of sleep apnea burden over a respective plurality of days;
determine a daily change in sleep apnea burden for the patient based on the determined plurality of classifications of sleep apnea burden over the respective plurality of days; and
determine at least one of a heart failure (HF) risk score, sudden cardiac arrest (SCA) risk score, arrhythmia risk score, or chronic obstructive pulmonary disease (COPD) risk score based, at least in part, on the daily change in sleep apnea burden.

12. The medical device claim 1, wherein the processing circuitry is further configured to:
determine heart rates of the patient based on the cardiac signal sensed during the period of time; and
detect the one or more sleep apnea episodes of the patient occurring during the period of time based on the heart rates.

13. A method comprising:
sensing, by sensing circuitry of a medical device, a cardiac signal indicating activity of a heart of a patient;
detecting, by processing circuitry, one or more sleep apnea episodes of the patient occurring during a period of time based on the cardiac signal;
comparing, by the processing circuitry, an amount of detected sleep apnea episodes of the patient during the period of time to a sleep apnea severity threshold; and
determining, by the processing circuitry, a classification of sleep apnea burden during the period of time based on the comparison.

14. The method of claim 13, further comprising:
determining, by the processing circuitry, the classification of sleep apnea burden is severe sleep apnea burden based on the amount of detected sleep apnea episodes being greater than a severe sleep apnea burden threshold.

15. The method of claim 13, further comprising:
determining, by the processing circuitry, at least one of a heart failure (HF) risk score, sudden cardiac arrest (SCA) risk score, arrhythmia risk score, or chronic obstructive pulmonary disease (COPD) risk score based, at least in part, on the classification of the sleep apnea burden.

16. The method of claim 13, further comprising:
determining, by the processing circuitry, at least one of a magnitude of heart rate change or a duration of heart rate change based on the cardiac signal;
classifying, by the processing circuitry, at least one of the detected sleep apnea episodes as obstructive sleep apnea or central sleep apnea based on at least one of the magnitude of heart rate change or the duration of heart rate change; and
determining, by the processing circuitry, at least one of a heart failure (HF) risk score, sudden cardiac arrest (SCA) risk score, arrhythmia risk score, or chronic obstructive pulmonary disease (COPD) risk score based, at least in part, on the classification of the sleep apnea burden and the classification of the at least one of the detected sleep apnea episodes as obstructive sleep apnea or central sleep apnea.

17. The method of claim 13, further comprising:
determining, by the processing circuitry, whether patient was non-complaint with continuous positive airway pressure (CPAP) during the period of time; and
determining, by the processing circuitry, at least one of a heart failure (HF) risk score, sudden cardiac arrest (SCA) risk score, arrhythmia risk score, or chronic obstructive pulmonary disease (COPD) risk score based, at least in part, on the classification of the sleep apnea burden and whether the patient was non-complaint with CPAP during the period of time.

18. The method of claim 13, further comprising:
determining, by the processing circuitry, a plurality of classifications of sleep apnea burden over a respective plurality of days;
determining, by the processing circuitry, a daily change in sleep apnea burden for the patient based on the determined plurality of classifications of sleep apnea burden over the respective plurality of days; and
determining, by the processing circuitry, at least one of a heart failure (HF) risk score, sudden cardiac arrest (SCA) risk score, arrhythmia risk score, or chronic obstructive pulmonary disease (COPD) risk score based, at least in part, on the daily change in sleep apnea burden.

19. The method of claim 13, further comprising:
determining, by the processing circuitry of the medical device, heart rates of the patient based on the cardiac signal sensed during the period of time; and
detecting, by the processing circuitry, the one or more sleep apnea episodes of the patient occurring during the period of time based on the heart rates.

20. A computer-readable medium comprising instructions that, when executed, causes processing circuitry of a medical system to:
sense a cardiac signal indicating activity of a heart of a patient;
detect one or more sleep apnea episodes of the patient occurring during a period of time based on the cardiac signal;
compare an amount of detected sleep apnea episodes of the patient during the period of time to a sleep apnea severity threshold; and
determine a classification of sleep apnea burden of the patient based on the comparison.
